(19) United States
(12) Patent Application Publication
(10) Pub. No.: US 2026/0167966 A1
(43) Pub. Date: Jun. 18, 2026

(54) METHOD FOR CIRCRNA CONSTRUCTION AND SYNTHESIS BASED ON THE COB GENE OF FUSARIUM OXYSPORUM AND RELATED APPLICATIONS

(71) Applicant: Bisheng (Beijing) Biotechnology Co., Ltd., Beijing (CN)

(72) Inventors: Daming WANG, Beijing (CN); Yongbo XUE, Beijing (CN); Hui LI, Beijing (CN)

(73) Assignee: Bisheng (Beijing) Biotechnology Co., Ltd., Beijing (CN)

(21) Appl. No.: 19/530,368

(22) Filed: Feb. 5, 2026

Related U.S. Application Data
(63) Continuation of application No. PCT/CN2025/088336, filed on Apr. 10, 2025.

(30) Foreign Application Priority Data
Jun. 21, 2024 (CN) 202410810975.4

(51) Int. Cl. C12N 15/113; C12P 19/34

(52) U.S. Cl. CPC C12N 15/113; C12P 19/34; C12N 2310/532

(57) ABSTRACT

The present disclosure relates to a construct of group I intron based on the cob gene of Fusarium oxysporum and a method for constructing the same, a method for RNA circularization, and a circular RNA and applications thereof, belonging to the field of biotechnology. The construct for in vitro RNA circularization provided uses a group I intron derived from the cob gene of Fusarium oxysporum as a self-cleaving ribozyme to mediate in vitro RNA circularization. Systematic analysis of the structure of cob group I intron demonstrated that P1 is necessary and unchangeable element for circularization while P10 and exons are not. Thus, the construct can effectively solve the problem of immune response caused by the large residual exogenous sequence in the existing in vitro synthesis of circularized RNA, and has promising application prospects.

### CROSS-REFERENCE TO RELATED APPLICATION

The present application is a continuation of international application of PCT application serial no. PCT/CN2025/088336, filed on Apr. 10, 2025, which claims the benefit of priority of Chinese Patent Application No. 202410810975.4, filed Jun. 21, 2024, and entitled “CONSTRUCT OF GROUP I INTRON BASED ON A COB GENE OF FUSARIUM OXYSPORUM AND METHOD FOR CONSTRUCTING THE SAME, METHOD FOR RNA CIRCULARIZATION, CIRCULAR RNA AND APPLICATIONS THEREOF,” the entire content of each of the above-mentioned patent applications is hereby incorporated herein by reference and made a part of this specification.

### REFERENCE TO A SEQUENCE LISTING

The instant application contains a Sequencing Listing which has been submitted electronically in XML file and is hereby incorporated by reference in its entirety. Said XML copy, created on Aug. 27, 2025, is named 159071_SEQUENCELISTING and is 55,077 bytes in size.

### TECHNICAL FIELD

The present disclosure belongs to the field of biotechnology, and in particular relates to a method for circRNA construction and synthesis based on the cob gene of Fusarium oxysporum and related applications.

### BACKGROUND OF RELATED ART

Circular RNA (circRNA) is one type of non-coding RNA molecules that does not have 5′-end cap and 3′ end poly(A) tail, but forms a circular structure with covalent bond. circRNA takes part in gene expression by regulating transcription initiation, elongation and alternative splicing, as well as serving as biomarkers of certain diseases.

Alternatively, circRNA has also been engineered to induce protein expression in the presence of internal ribosome entry site (IRES) transcription-initiation element. Different with canonical 5′cap-dependent initiation in linear mRNA translation, IRES mediates initiation in a cap-independent pathway by binding to distinct elongation transcription factors (eIFs).

At present, the most commonly used method for RNA circularization in vitro is by taking advantage of group I and group II self-splicing ribozymes, which are further reengineered to construct a Permutated Intron-Exon (PIE) system with gene of interest inserted. Group I ribozymes derived from T4 bacteriophage, Anabeana, and Tetrahymena are typically applied and show variable circularization efficiency. The PIE construct relies on an autocatalytic cleaving reaction of intron ribozymes, namely, the 5′ and 3′ introns interacting with each other and falling off during self-cleaving, and the exons on both ends are ligated to achieve in vitro circularization of the target sequence. However, when the target sequence is internalized in circRNA, there will be a residue of a long exogenous sequence (i.g. scar sequence). In clinical therapeutics, the residual exogenous sequence may cause greater unexpected immunogenicity when delivered in vivo, resulting in reduced expression of the target protein, which has great limitations.

### SUMMARY OF THE INVENTION

A first object of the present disclosure is to provide a construct for in vitro RNA synthesis and circularization. When the construct is applied for in vitro circRNA synthesis, it exhibits distinguished circularization efficiency even in the absence of exons, leading to extremely low extraneous sequence with only 5 nt in final circRNA product. This design, to a large degree, enriches the existing circRNA synthesis methods, and overcomes the defects of long exogenous sequence residue.

A second object of the present disclosure is to provide a method for constructing a construct for in vitro circRNA synthesis.

A third object of the present disclosure is to provide a construct obtained by the method for constructing a construct for in vitro RNA synthesis as described above.

A fourth object of the present disclosure is to provide a method for RNA circularization.

A fifth object of the present disclosure is to provide a circular RNA.

A sixth object of the present disclosure is to provide applications of the circular RNA described above in the preparation of medicines, vaccines and/or cells highly expressing target genes.

Specifically, the construct for in vitro circRNA synthesis provided by the present disclosure, following the 5′-3′ direction, includes 3′-intron, exon 2, gene of interest, exon 1, and 5′-intron; among them, the 3′-intron and the 5′-intron are derived from a group I intron of a cob gene of Fusarium oxysporum with a nucleotide sequence as shown in SEQ ID NO: 1, and the 5′-intron contains a P1 domain, and the P1 domain comprises a nucleotide fragment with a sequence as shown in SEQ ID NO: 38; the exon 1 is an exon fragment adjacent to the 5′ end of the group I intron, and the length of the exon 1 is 0-393 nt; the exon 2 is an exon fragment adjacent to the 3′ end of the group I intron, and the length of the exon 2 is 0-780 nt.

Further, the nucleotide sequence of the group I intron is as shown in SEQ ID NO: 2.

Further, the 5′-intron and 3′-intron are obtained by splitting the group I intron, and splitting sites on the group I intron are located at positions 18-1020 of a sequence as shown in SEQ ID NO: 2.

Further, the nucleotide sequence of the 3′-intron is shown as in SEQ ID NO: 29, the nucleotide sequence of the 5′-intron is shown as in SEQ ID NO: 6, and the lengths of exon 1 and exon 2 are 0 nt.

Further, the construct includes a T7 promoter, and the T7 promoter is located at the 5′ end of the 3′-intron.

Further, the nucleotide sequence of the T7 promoter is shown as in SEQ ID NO: 11.

Further, the construct includes a CVB3 IRES sequence, and the CVB3 IRES sequence is located between the gene of interest and the exon 1.

Further, the nucleotide sequence of the CVB3 IRES sequence is as shown in SEQ ID NO: 13.

The method for constructing a construct for in vitro RNA circularization provided by the present disclosure includes: performing a structural analysis on a group I intron of a cob gene of Fusarium oxysporum to obtain splitting sites thereby making different constructs; introducing a gene of interest into the construct framework to complete the construct.

Further, the nucleotide sequence of the group I intron is shown as in SEQ ID NO: 2.

Further, the splitting sites locate at positions 18-1020 of the sequence as shown in SEQ ID NO: 2.

The present disclosure provides a construct obtained by the method for making a construct for in vitro circRNA synthesis and circularization is as described above.

Further, the construct, following the 5′-3′ direction, includes 3′-intron, exon 2, gene of interest, exon 1 and 5′-intron.

Further, the nucleotide sequence of the 3′-intron is shown as in SEQ ID NO: 29, the nucleotide sequence of the 5′-intron is shown as in SEQ ID NO: 6, and the lengths of exon 1 and exon 2 are 0 nt.

The method for RNA circularization provided by the present disclosure includes: taking the preceding construct for transcription to obtain a linear RNA molecule; and inducing the linear RNA molecule for circularization to obtain the circular RNA.

The circular RNA provided by the present disclosure is prepared by the preceding method for RNA circularization.

The present disclosure further provides applications of the construct and/or circular RNA described above in the preparation of medicines, vaccines and/or cells highly expressing target genes.

### Beneficial Effects

The construct for in vitro circRNA synthesis and circularization provided by the present disclosure uses the group I intron derived from the cob gene of Fusarium oxysporum as a self-cleaving ribozyme that mediates in vitro RNA circularization. The construct effectively catalyzes the in vitro RNA circularization with or without the introduction of a small amount of exon fragments, and only 5 nt of exogenous sequence remains in the final obtained circular RNA. Thus, the construct effectively addresses the issue of immune response caused by the existing approaches of in vitro circRNA synthesis, and has promising application prospects.

### BRIEF DESCRIPTION OF THE DRAWINGS

FIG. 1. Schematic diagram of the cob gene components provided in Embodiment 1 of the present disclosure.

FIG. 2. Schematic diagram of a predicted secondary structure of a group I intron provided in Embodiment 1 of the present disclosure.

FIG. 3. Schematic representation of PIE design of the construct and its circular RNA provided in Embodiment 1 of the present disclosure.

FIG. 4. CircGFP analysis using urea-PAGE gel electrophoresis provided in Embodiment 1 of the present disclosure.

FIG. 5. Sanger sequencing result diagram of circular RNA and alignment with putative sequence provided in Embodiment 1 of the present disclosure.

FIG. 6. Schematic diagram of stepwise deletion of nucleotides in the initial exon 2 provided in Embodiment 2 of the present disclosure.

FIG. 7. Schematic diagram of stepwise deletion of nucleotides in the initial exon 1 provided in Embodiment 2 of the present disclosure.

FIG. 8. CircGFP analysis using urea-PAGE gel electrophoresis provided in Embodiment 2 of the present disclosure (in vitro transcription).

FIG. 9. Experimental result diagram of urea-PAGE gel electrophoresis provided in Embodiment 2 of the present disclosure (in vitro circularization).

FIG. 10. Predicted secondary structure of the group I intron P1 domain provided in the present disclosure.

FIG. 11. Base sequences and secondary structures of the group I intron P9 and P10 domains provided in the present disclosure.

FIG. 12. Schematic diagram and Sanger sequencing of modifications of F2-3′-intron provided in Embodiment 3 of the present disclosure.

FIG. 13. Schematic diagram and Sanger sequencing of a modifications of F2-5′-intron provided in Embodiment 3 of the present disclosure.

FIG. 14. CircGFP analysis using urea-PAGE gel electrophoresis provided in Embodiment 3 of the present disclosure (constructs 11-13);

FIG. 15. Sanger sequencing result diagram of reverse-transcribed circular RNA 11 and its alignment with putative sequence provided in Embodiment 3 of the present disclosure.

FIG. 16. Schematic diagram and Sanger sequencing of a modifications of F2-5′-intron provided in Embodiment 3 of the present disclosure (mutating the P1 domain).

FIG. 17. CircGFP analysis using urea-PAGE gel electrophoresis provided in Embodiment 3 of the present disclosure (constructs 14-16);

FIG. 18. CircGFP analysis using urea-PAGE gel electrophoresis provided in Embodiment 4 of the present disclosure.

### DETAILED DESCRIPTION OF THE EMBODIMENTS

The nucleotide sequences involved in the present disclosure are as shown in Table 1:

TABLE 1

| Description | SEQ ID NO: | Sequence |
|---|---|---|
| cob gene | 1 | ATGAGAATATTAAAAAATCACCCTTTATTAAAATTAGCTAATGGT |
| | | TATTTAATAGACGCTTCACAACCTAGTAATATAAGTTACTTATGA |
| | | AATTTCGGATCTTTATTATTAGTTTGTTTAGTTATACAAATTGTA |
| | | ACTGGAGTTACTTTAGCTATGCATTATAACCCTTCAGTATTAGAA |
| | | GCATTTAATTCTGTAGAACATATTATGCGTGATGTAAACAATGGA |
| | | TGATTAGTACGTTACTTACATAGTAACACAGCATCAGCTTTCTTC |
| | | TTTTTAGTGTACTTACACATAGGAAGAGGTATATATTACGCATCT |
| | | TATAGAGCACCAAGAACATTGACATGAGTTATAGGTACAATAATC |
| | | CTTATCGTTATGATAGTTACAGGATTCCTGGGTTACCATACAATA |
| | | GCCCAAAACGACTATAATAATAATAAAATAACAACTACAACAACC |
| | | TTTAACGATAAAAGATATTATTCAACATCGAGAAATAATGAAGAG |
| | | GAGACTTCGTTCTCACGTATAAATAAGTTTTTATTAGCCAAAAAT |
| | | CTTAATCCTGTTTTTATCTATCATAATCTAAATGAAGATTCAGTT |
| | | CGTAGAAATATAGCTAAAGAAACTAAGGGACTTAGTGGTATTTAT |
| | | ATGATCTTAAATAAAGAAACTTTAAGTTATTATATTGGATCAGCT |
| | | TCTACTGACAGAATTAACTCTAGATTTTCAAAACATTTAATATAT |
| | | TTAAATGGTAGTAAAATAGTTAAAAATTCAGTTAATAAATATGGT |
| | | TTACATAATTTTGCCTTTATTGTATTAGAATTATTCCCTGAAATA |
| | | GTTAATCAAGAAAATAATAAAAAATTATTAGATTTAGAAGATTTT |
| | | TATCTAAAATCTCTTTTACCTGACTATAATATATTAACCGAAGCA |
| | | GGATCTAGCTTTGGATATAAACATACTGAAGTTAATAGAATAAAG |
| | | ATGAAAGCTAATTATAGTGAGAAGCGTAGAGAAGAAATAGGTAGT |
| | | TTGAATAGAGGTAAAACTTTATCTTCTGAAACTATAGAAACTATG |
| | | AGACAATCAGCTTTAAATAGAAAACCTTTAGACTATACAGAACAA |
| | | GGTGTTTTAAATATGAAAAAGAATTCTAAGCCTATTATAGTAAAA |
| | | GAATTAAATAATACTGTATATGGCGAATTTAATAGTATAGTTGAA |
| | | GCAGCAGAAGCTTTAAATTGTTCAACTAAAACTATACAAAGAACA |
| | | TTAAAAACTCCTAGCAAAACATTAAAAGGACGTTGAATAGTTGAT |
| | | TATTTTAAATAAGGGGCAAGCTCTAAAATAAATTATTATTATAGT |
| | | TGTAGTCCTGCGAGTATAAAGTTTTATGCAATTTATGGAAAGAAA |
| | | TAAAACTTAAAGCAGAATAACCTGACAAGGTTATTGAAGAAACCT |
| | | AATAGTTTCTTTGGCAATATTAGTGAAAACGATCAAAATATATTG |
| | | TAACTACAATATAAGAGATCGTCGGTTATCCATATAATCGCGACA |
| | | GACTGGGTCACTAATGGGTGGCTGAAATGCTGCTTAATGCACAGT |
| | | CGAAACTTTATATCAAGGCGATATATATCGCTCTAATATAGGTGA |
| | | TAAACGAACAATAAAGTTATCATCGCTTTTGTACTTTGCTACAAA |
| | | AGTAAGTTTGTATGTTTTACCTTATGGACAAATGTCATTATGAGG |
| | | TGCTACAGTTATTACTAATTTAATTAGTGCTGTTCCTTGAATTGG |
| | | ACAAGACATAGTTGAGTTCATTTGAGGAGGTTTTAGCGTAAATAA |
| | | CGCAACTTTAAACAGATTCTTTGCTTTACATTTTGTATTACCTTT |
| | | TATATTAGCGGCATTAGTTTTAATGCATATGATAGCTTTACACGA |
| | | TACAGCTGGATCAAGTAATCCTTTAGGAGTACCAGTATATTATGA |
| | | TAGAATGCCTATGGCTCCATATTTCTTATTTAAAGATTTAATAAC |
| | | TATATTTATCTTTATATTTGTTTTAGGTATCTTTGTATTCTTTAT |
| | | GCCTAATGTTTTAGGTGATAGTGATAATTATATAATGGCTAACCC |
| | | TATGCAAACACCACCTGCGATAGTACCTGAATGATATTTATTACC |
| | | TTTCTATGCTATTTTAAGATCTATCCCTAATAAATTATTAGGAGT |
| | | TATCGCAATGTTAGCTGCAATACTAATTATTTTACTATTACCTAA |
| | | AGCTGATTTAGGGTTAACTAAAGGTTTACAATTTAGACCCCTAAG |
| | | CAAGATAGCTTTCTACCTGTTTTTAGTGAATTTTTTAATGTTAAT |
| | | GCAATTAGGGGCTAAACACGTAGAAAGCCCGTTTATAGAAATAGG |
| | | ACAATTAAGTACTGCGTTATATTTTTCTCATTTCTTATTTATATT |
| | | ACCGGCAGTAAGTATATTAGAAAATACTCTTGTAGATTTAGAATT |
| | | ACAAAATAATGCCAAAAATATATAA |
| Group I intron | 2 | TACCATACAATAGCCCAAAACGACTATAATAATAATAAAATAACA |
| | | ACTACAACAACCTTTAACGATAAAAGATATTATTCAACATCGAGA |
| | | AATAATGAAGAGGAGACTTCGTTCTCACGTATAAATAAGTTTTTA |
| | | TTAGCCAAAAATCTTAATCCTGTTTTTATCTATCATAATCTAAAT |
| | | GAAGATTCAGTTCGTAGAAATATAGCTAAAGAAACTAAGGGACTT |
| | | AGTGGTATTTATATGATCTTAAATAAAGAAACTTTAAGTTATTAT |
| | | ATTGGATCAGCTTCTACTGACAGAATTAACTCTAGATTTTCAAAA |
| | | CATTTAATATATTTAAATGGTAGTAAAATAGTTAAAAATTCAGTT |
| | | AATAAATATGGTTTACATAATTTTGCCTTTATTGTATTAGAATTA |
| | | TTCCCTGAAATAGTTAATCAAGAAAATAATAAAAAATTATTAGAT |
| | | TTAGAAGATTTTTATCTAAAATCTCTTTTACCTGACTATAATATA |
| | | TTAACCGAAGCAGGATCTAGCTTTGGATATAAACATACTGAAGTT |
| | | AATAGAATAAAGATGAAAGCTAATTATAGTGAGAAGCGTAGAGAA |
| | | GAAATAGGTAGTTTGAATAGAGGTAAAACTTTATCTTCTGAAACT |
| | | ATAGAAACTATGAGACAATCAGCTTTAAATAGAAAACCTTTAGAC |
| | | TATACAGAACAAGGTGTTTTAAATATGAAAAAGAATTCTAAGCCT |
| | | ATTATAGTAAAAGAATTAAATAATACTGTATATGGCGAATTTAAT |
| | | AGTATAGTTGAAGCAGCAGAAGCTTTAAATTGTTCAACTAAAACT |
| | | ATACAAAGAACATTAAAAACTCCTAGCAAAACATTAAAAGGACGT |
| | | TGAATAGTTGATTATTTTAAATAAGGGGCAAGCTCTAAAATAAAT |
| | | TATTATTATAGTTGTAGTCCTGCGAGTATAAAGTTTTATGCAATT |
| | | TATGGAAAGAAATAAAACTTAAAGCAGAATAACCTGACAAGGTTA |
| | | TTGAAGAAACCTAATAGTTTCTTTGGCAATATTAGTGAAAACGAT |
| | | CAAAATATATTGTAACTACAATATAAGAGATCGTCGGTTATCCAT |
| | | ATAATCGCGACAGACTGGGTCACTAATGGGTGGCTGAAATGCTGC |
| | | TTAATGCACAGTCGAAACTTTATATCAAGGCGATATATATCGCTC |
| | | TAATATAGGTGATAAACGAACAATAAAGTTATCATCGCTTTTGTA |
| | | CTTTGCTACAAAAGTAAGTTTG |
| F1-3′-intron | 3 | TTCGTTCTCACGTATAAATAAGTTTTTATTAGCCAAAAATCTTAA |
| | | TCCTGTTTTTATCTATCATAATCTAAATGAAGATTCAGTTCGTAG |
| | | AAATATAGCTAAAGAAACTAAGGGACTTAGTGGTATTTATATGAT |
| | | CTTAAATAAAGAAACTTTAAGTTATTATATTGGATCAGCTTCTAC |
| | | TGACAGAATTAACTCTAGATTTTCAAAACATTTAATATATTTAAA |
| | | TGGTAGTAAAATAGTTAAAAATTCAGTTAATAAATATGGTTTACA |
| | | TAATTTTGCCTTTATTGTATTAGAATTATTCCCTGAAATAGTTAA |
| | | TCAAGAAAATAATAAAAAATTATTAGATTTAGAAGATTTTTATCT |
| | | AAAATCTCTTTTACCTGACTATAATATATTAACCGAAGCAGGATC |
| | | TAGCTTTGGATATAAACATACTGAAGTTAATAGAATAAAGATGAA |
| | | AGCTAATTATAGTGAGAAGCGTAGAGAAGAAATAGGTAGTTTGAA |
| | | TAGAGGTAAAACTTTATCTTCTGAAACTATAGAAACTATGAGACA |
| | | ATCAGCTTTAAATAGAAAACCTTTAGACTATACAGAACAAGGTGT |
| | | TTTAAATATGAAAAAGAATTCTAAGCCTATTATAGTAAAAGAATT |
| | | AAATAATACTGTATATGGCGAATTTAATAGTATAGTTGAAGCAGC |
| | | AGAAGCTTTAAATTGTTCAACTAAAACTATACAAAGAACATTAAA |
| | | AACTCCTAGCAAAACATTAAAAGGACGTTGAATAGTTGATTATTT |
| | | TAAATAAGGGGCAAGCTCTAAAATAAATTATTATTATAGTTGTAG |
| | | TCCTGCGAGTATAAAGTTTTATGCAATTTATGGAAAGAAATAAAA |
| | | CTTAAAGCAGAATAACCTGACAAGGTTATTGAAGAAACCTAATAG |
| | | TTTCTTTGGCAATATTAGTGAAAACGATCAAAATATATTGTAACT |
| | | ACAATATAAGAGATCGTCGGTTATCCATATAATCGCGACAGACTG |
| | | GGTCACTAATGGGTGGCTGAAATGCTGCTTAATGCACAGTCGAAA |
| | | CTTTATATCAAGGCGATATATATCGCTCTAATATAGGTGATAAAC |
| | | GAACAATAAAGTTATCATCGCTTTTGTACTTTGCTACAAAAGTAA |
| | | GTTTGTATGTT |
| F1-5′-intron | 4 | TGGGTTACCATACAATAGCCCAAAACGACTATAATAATAATAAAA |
| | | TAACAACTACAACAACCTTTAACGATAAAAGATATTATTCAACAT |
| | | CGAGAAATAATGAAGAGGAGAC |
| F2-3′-intron | 5 | AAATCTTAATCCTGTTTTTATCTATCATAATCTAAATGAAGATTC |
| | | AGTTCGTAGAAATATAGCTAAAGAAACTAAGGGACTTAGTGGTAT |
| | | TTATATGATCTTAAATAAAGAAACTTTAAGTTATTATATTGGATC |
| | | AGCTTCTACTGACAGAATTAACTCTAGATTTTCAAAACATTTAAT |
| | | ATATTTAAATGGTAGTAAAATAGTTAAAAATTCAGTTAATAAATA |
| | | TGGTTTACATAATTTTGCCTTTATTGTATTAGAATTATTCCCTGA |
| | | AATAGTTAATCAAGAAAATAATAAAAAATTATTAGATTTAGAAGA |
| | | TTTTTATCTAAAATCTCTTTTACCTGACTATAATATATTAACCGA |
| | | AGCAGGATCTAGCTTTGGATATAAACATACTGAAGTTAATAGAAT |
| | | AAAGATGAAAGCTAATTATAGTGAGAAGCGTAGAGAAGAAATAGG |
| | | TAGTTTGAATAGAGGTAAAACTTTATCTTCTGAAACTATAGAAAC |
| | | TATGAGACAATCAGCTTTAAATAGAAAACCTTTAGACTATACAGA |
| | | ACAAGGTGTTTTAAATATGAAAAAGAATTCTAAGCCTATTATAGT |
| | | AAAAGAATTAAATAATACTGTATATGGCGAATTTAATAGTATAGT |
| | | TGAAGCAGCAGAAGCTTTAAATTGTTCAACTAAAACTATACAAAG |
| | | AACATTAAAAACTCCTAGCAAAACATTAAAAGGACGTTGAATAGT |
| | | TGATTATTTTAAATAAGGGGCAAGCTCTAAAATAAATTATTATTA |
| | | TAGTTGTAGTCCTGCGAGTATAAAGTTTTATGCAATTTATGGAAA |
| | | GAAATAAAACTTAAAGCAGAATAACCTGACAAGGTTATTGAAGAA |
| | | ACCTAATAGTTTCTTTGGCAATATTAGTGAAAACGATCAAAATAT |
| | | ATTGTAACTACAATATAAGAGATCGTCGGTTATCCATATAATCGC |
| | | GACAGACTGGGTCACTAATGGGTGGCTGAAATGCTGCTTAATGCA |
| | | CAGTCGAAACTTTATATCAAGGCGATATATATCGCTCTAATATAG |
| | | GTGATAAACGAACAATAAAGTTATCATCGCTTTTGTACTTTGCTA |
| | | CAAAAGTAAGTTTGTATGTT |
| F2-5′-intron | 6 | TGGGTTACCATACAATAGCCCAAAACGACTATAATAATAATAAAA |
| | | TAACAACTACAACAACCTTTAACGATAAAAGATATTATTCAACAT |
| | | CGAGAAATAATGAAGAGGAGACTTCGTTCTCACGTATAAATAAGT |
| | | TTTTATTAGCCAA |
| F3-3′-intron | 7 | TAAATAATACTGTATATGGCGAATTTAATAGTATAGTTGAAGCAG |
| | | CAGAAGCTTTAAATTGTTCAACTAAAACTATACAAAGAACATTAA |
| | | AAACTCCTAGCAAAACATTAAAAGGACGTTGAATAGTTGATTATT |
| | | TTAAATAAGGGGCAAGCTCTAAAATAAATTATTATTATAGTTGTA |
| | | GTCCTGCGAGTATAAAGTTTTATGCAATTTATGGAAAGAAATAAA |
| | | ACTTAAAGCAGAATAACCTGACAAGGTTATTGAAGAAACCTAATA |
| | | GTTTCTTTGGCAATATTAGTGAAAACGATCAAAATATATTGTAAC |
| | | TACAATATAAGAGATCGTCGGTTATCCATATAATCGCGACAGACT |
| | | GGGTCACTAATGGGTGGCTGAAATGCTGCTTAATGCACAGTCGAA |
| | | ACTTTATATCAAGGCGATATATATCGCTCTAATATAGGTGATAAA |
| | | CGAACAATAAAGTTATCATCGCTTTTGTACTTTGCTACAAAAGTA |
| | | AGTTTGTATGTT |
| F3-5′-intron | 8 | TGGGTTACCATACAATAGCCCAAAACGACTATAATAATAATAAAA |
| | | TAACAACTACAACAACCTTTAACGATAAAAGATATTATTCAACAT |
| | | CGAGAAATAATGAAGAGGAGACTTCGTTCTCACGTATAAATAAGT |
| | | TTTTATTAGCCAAAAATCTTAATCCTGTTTTTATCTATCATAATC |
| | | TAAATGAAGATTCAGTTCGTAGAAATATAGCTAAAGAAACTAAGG |
| | | GACTTAGTGGTATTTATATGATCTTAAATAAAGAAACTTTAAGTT |
| | | ATTATATTGGATCAGCTTCTACTGACAGAATTAACTCTAGATTTT |
| | | CAAAACATTTAATATATTTAAATGGTAGTAAAATAGTTAAAAATT |
| | | CAGTTAATAAATATGGTTTACATAATTTTGCCTTTATTGTATTAG |
| | | AATTATTCCCTGAAATAGTTAATCAAGAAAATAATAAAAAATTAT |
| | | TAGATTTAGAAGATTTTTATCTAAAATCTCTTTTACCTGACTATA |
| | | ATATATTAACCGAAGCAGGATCTAGCTTTGGATATAAACATACTG |
| | | AAGTTAATAGAATAAAGATGAAAGCTAATTATAGTGAGAAGCGTA |
| | | GAGAAGAAATAGGTAGTTTGAATAGAGGTAAAACTTTATCTTCTG |
| | | AAACTATAGAAACTATGAGACAATCAGCTTTAAATAGAAAACCTT |
| | | TAGACTATACAGAACAAGGTGTTTTAAATATGAAAAAGAATTCTA |
| | | AGCCTATTATAGTAAAAGAAT |
| initial exon 1 | 9 | CGCATCTTATAGAGCACCAAGAACATTGACATGAGTTATAGGTAC |
| | | AATAATCCTTATCGTTATGATAGTTACAGGATTCC |
| initial exon 2 | 10 | TTACCTTATGGACAAATGTCATTATGAGGTGCTACAGTTATTACT |
| | | AATTTAATTAGTGCTGTTCCTTGAATTGGACAAGA |
| T7 promoter | 11 | TAATACGACTCACTATAGGG |
| GFP gene | 12 | ATGCCCGCCATGAAGATCGAGTGCCGCATCACCGGCACCCTGAAC |
| | | GGCGTGGAGTTCGAGCTGGTGGGCGGCGGAGAGGGCACCCCCGAG |
| | | CAGGGCCGCATGACCAACAAGATGAAGAGCACCAAAGGCGCCCTG |
| | | ACCTTCAGCCCCTACCTGCTGAGCCACGTGATGGGCTACGGCTTC |
| | | TACCACTTCGGCACCTACCCCAGCGGCTACGAGAACCCCTTCCTG |
| | | CACGCCATCAACAACGGCGGCTACACCAACACCCGCATCGAGAAG |
| | | TACGAGGACGGCGGCGTGCTGCACGTGAGCTTCAGCTACCGCTAC |
| | | GAGGCCGGCCGCGTGATCGGCGACTTCAAGGTGGTGGGCACCGGC |
| | | TTCCCCGAGGACAGCGTGATCTTCACCGACAAGATCATCCGCAGC |
| | | AACGCCACCGTGGAGCACCTGCACCCCATGGGCGATAACGTGCTG |
| | | GTGGGCAGCTTCGCCCGCACCTTCAGCCTGCGCGACGGCGGCTAC |
| | | TACAGCTTCGTGGTGGACAGCCACATGCACTTCAAGAGCGCCATC |
| | | CACCCCAGCATCCTGCAGAACGGGGGCCCCATGTTCGCCTTCCGC |
| | | CGCGTGGAGGAGCTGCACAGCAACACCGAGCTGGGCATCGTGGAG |
| | | TACCAGCACGCCTTCAAGACCCCCATCGCCTTCGCCAGATCTCGA |
| | | GCTCGATGATAA |
| CVB3 IRES | 13 | TTAAAACAGCCTGTGGGTTGATCCCACCCACAGGCCCATTG |
| sequence | | GGCGCTAGCACTCTGGTATCACGGTACCTTTGTGCGCCTGTTTTA |
| | | TACCCCCTCCCCCAACTGTAACTTAGAAGTAACACACACCGATCA |
| | | ACAGTCAGCGTGGCACACCAGCCACGTTTTGATCAAGCACTTCTG |
| | | TTACCCCGGACTGAGTATCAATAGACTGCTCACGCGGTTGAAGGA |
| | | GAAAGCGTTCGTTATCCGGCCAACTACTTCGAAAAACCTAGTAAC |
| | | ACCGTGGAAGTTGCAGAGTGTTTCGCTCAGCACTACCCCAGTGTA |
| | | GATCAGGTCGATGAGTCACCGCATTCCCCACGGGCGACCGTGGCG |
| | | GTGGCTGCGTTGGCGGCCTGCCCATGGGGAAACCCATGGGACGCT |
| | | CTAATACAGACATGGTGCGAAGAGTCTATTGAGCTAGTTGGTAGT |
| | | CCTCCGGCCCCTGAATGCGGCTAATCCTAACTGCGGAGCACACAC |
| | | CCTCAAGCCAGAGGGCAGTGTGTCGTAACGGGCAACTCTGCAGCG |
| | | GAACCGACTACTTTGGGTGTCCGTGTTTCATTTTATTCCTATACT |
| | | GGCTGCTTATGGTGACAATTGAGAGATCGTTACCATATAGCTATT |
| | | GGATTGGCCATCCGGTGACTAATAGAGCTATTATATATCCCTTTG |
| | | TTGGGTTTATACCACTTAGCTTGAAAGAGGTTAAAACATTACAAT |
| | | TCATTGTTAAGTTGAATACAGCAAA |
| Construct 1 | 14 | TAATACGACTCACTATAGGGTTCGTTCTCACGTATAAATAAGTTT |
| | | TTATTAGCCAAAAATCTTAATCCTGTTTTTATCTATCATAATCTA |
| | | AATGAAGATTCAGTTCGTAGAAATATAGCTAAAGAAACTAAGGGA |
| | | CTTAGTGGTATTTATATGATCTTAAATAAAGAAACTTTAAGTTAT |
| | | TATATTGGATCAGCTTCTACTGACAGAATTAACTCTAGATTTTCA |
| | | AAACATTTAATATATTTAAATGGTAGTAAAATAGTTAAAAATTCA |
| | | GTTAATAAATATGGTTTACATAATTTTGCCTTTATTGTATTAGAA |
| | | TTATTCCCTGAAATAGTTAATCAAGAAAATAATAAAAAATTATTA |
| | | GATTTAGAAGATTTTTATCTAAAATCTCTTTTACCTGACTATAAT |
| | | ATATTAACCGAAGCAGGATCTAGCTTTGGATATAAACATACTGAA |
| | | GTTAATAGAATAAAGATGAAAGCTAATTATAGTGAGAAGCGTAGA |
| | | GAAGAAATAGGTAGTTTGAATAGAGGTAAAACTTTATCTTCTGAA |
| | | ACTATAGAAACTATGAGACAATCAGCTTTAAATAGAAAACCTTTA |
| | | GACTATACAGAACAAGGTGTTTTAAATATGAAAAAGAATTCTAAG |
| | | CCTATTATAGTAAAAGAATTAAATAATACTGTATATGGCGAATTT |
| | | AATAGTATAGTTGAAGCAGCAGAAGCTTTAAATTGTTCAACTAAA |
| | | ACTATACAAAGAACATTAAAAACTCCTAGCAAAACATTAAAAGGA |
| | | CGTTGAATAGTTGATTATTTTAAATAAGGGGCAAGCTCTAAAATA |
| | | AATTATTATTATAGTTGTAGTCCTGCGAGTATAAAGTTTTATGCA |
| | | ATTTATGGAAAGAAATAAAACTTAAAGCAGAATAACCTGACAAGG |
| | | TTATTGAAGAAACCTAATAGTTTCTTTGGCAATATTAGTGAAAAC |
| | | GATCAAAATATATTGTAACTACAATATAAGAGATCGTCGGTTATC |
| | | CATATAATCGCGACAGACTGGGTCACTAATGGGTGGCTGAAATGC |
| | | TGCTTAATGCACAGTCGAAACTTTATATCAAGGCGATATATATCG |
| | | CTCTAATATAGGTGATAAACGAACAATAAAGTTATCATCGCTTTT |
| | | GTACTTTGCTACAAAAGTAAGTTTGTATGTTTTACCTTATGGACA |
| | | AATGTCATTATGAGGTGCTACAGTTATTACTAATTTAATTAGTGC |
| | | TGTTCCTTGAATTGGACAAGAGCCACCATGCCCGCCATGAAGATC |
| | | GAGTGCCGCATCACCGGCACCCTGAACGGCGTGGAGTTCGAGCTG |
| | | GTGGGCGGCGGAGAGGGCACCCCCGAGCAGGGCCGCATGACCAAC |
| | | AAGATGAAGAGCACCAAAGGCGCCCTGACCTTCAGCCCCTACCTG |
| | | CTGAGCCACGTGATGGGCTACGGCTTCTACCACTTCGGCACCTAC |
| | | CCCAGCGGCTACGAGAACCCCTTCCTGCACGCCATCAACAACGGC |
| | | GGCTACACCAACACCCGCATCGAGAAGTACGAGGACGGCGGCGTG |
| | | CTGCACGTGAGCTTCAGCTACCGCTACGAGGCCGGCCGCGTGATC |
| | | GGCGACTTCAAGGTGGTGGGCACCGGCTTCCCCGAGGACAGCGTG |
| | | ATCTTCACCGACAAGATCATCCGCAGCAACGCCACCGTGGAGCAC |
| | | CTGCACCCCATGGGCGATAACGTGCTGGTGGGCAGCTTCGCCCGC |
| | | ACCTTCAGCCTGCGCGACGGCGGCTACTACAGCTTCGTGGTGGAC |
| | | AGCCACATGCACTTCAAGAGCGCCATCCACCCCAGCATCCTGCAG |
| | | AACGGGGGCCCCATGTTCGCCTTCCGCCGCGTGGAGGAGCTGCAC |
| | | AGCAACACCGAGCTGGGCATCGTGGAGTACCAGCACGCCTTCAAG |
| | | ACCCCCATCGCCTTCGCCAGATCTCGAGCTCGATGATAATTAAAA |
| | | CAGCCTGTGGGTTGATCCCACCCACAGGCCCATTGGGCGCTAGCA |
| | | CTCTGGTATCACGGTACCTTTGTGCGCCTGTTTTATACCCCCTCC |
| | | CCCAACTGTAACTTAGAAGTAACACACACCGATCAACAGTCAGCG |
| | | TGGCACACCAGCCACGTTTTGATCAAGCACTTCTGTTACCCCGGA |
| | | CTGAGTATCAATAGACTGCTCACGCGGTTGAAGGAGAAAGCGTTC |
| | | GTTATCCGGCCAACTACTTCGAAAAACCTAGTAACACCGTGGAAG |
| | | TTGCAGAGTGTTTCGCTCAGCACTACCCCAGTGTAGATCAGGTCG |
| | | ATGAGTCACCGCATTCCCCACGGGCGACCGTGGCGGTGGCTGCGT |
| | | TGGCGGCCTGCCCATGGGGAAACCCATGGGACGCTCTAATACAGA |
| | | CATGGTGCGAAGAGTCTATTGAGCTAGTTGGTAGTCCTCCGGCCC |
| | | CTGAATGCGGCTAATCCTAACTGCGGAGCACACACCCTCAAGCCA |
| | | GAGGGCAGTGTGTCGTAACGGGCAACTCTGCAGCGGAACCGACTA |
| | | CTTTGGGTGTCCGTGTTTCATTTTATTCCTATACTGGCTGCTTAT |
| | | GGTGACAATTGAGAGATCGTTACCATATAGCTATTGGATTGGCCA |
| | | TCCGGTGACTAATAGAGCTATTATATATCCCTTTGTTGGGTTTAT |
| | | ACCACTTAGCTTGAAAGAGGTTAAAACATTACAATTCATTGTTAA |
| | | GTTGAATACAGCAAACGCATCTTATAGAGCACCAAGAACATTGAC |
| | | ATGAGTTATAGGTACAATAATCCTTATCGTTATGATAGTTACAGG |
| | | ATTCCTGGGTTACCATACAATAGCCCAAAACGACTATAATAATAA |
| | | TAAAATAACAACTACAACAACCTTTAACGATAAAAGATATTATTC |
| | | AACATCGAGAAATAATGAAGAGGAGAC |
| Construct 2 | 15 | TAATACGACTCACTATAGGGAAATCTTAATCCTGTTTTTATCTAT |
| | | CATAATCTAAATGAAGATTCAGTTCGTAGAAATATAGCTAAAGAA |
| | | ACTAAGGGACTTAGTGGTATTTATATGATCTTAAATAAAGAAACT |
| | | TTAAGTTATTATATTGGATCAGCTTCTACTGACAGAATTAACTCT |
| | | AGATTTTCAAAACATTTAATATATTTAAATGGTAGTAAAATAGTT |
| | | AAAAATTCAGTTAATAAATATGGTTTACATAATTTTGCCTTTATT |
| | | GTATTAGAATTATTCCCTGAAATAGTTAATCAAGAAAATAATAAA |
| | | AAATTATTAGATTTAGAAGATTTTTATCTAAAATCTCTTTTACCT |
| | | GACTATAATATATTAACCGAAGCAGGATCTAGCTTTGGATATAAA |
| | | CATACTGAAGTTAATAGAATAAAGATGAAAGCTAATTATAGTGAG |
| | | AAGCGTAGAGAAGAAATAGGTAGTTTGAATAGAGGTAAAACTTTA |
| | | TCTTCTGAAACTATAGAAACTATGAGACAATCAGCTTTAAATAGA |
| | | AAACCTTTAGACTATACAGAACAAGGTGTTTTAAATATGAAAAAG |
| | | AATTCTAAGCCTATTATAGTAAAAGAATTAAATAATACTGTATAT |
| | | GGCGAATTTAATAGTATAGTTGAAGCAGCAGAAGCTTTAAATTGT |
| | | TCAACTAAAACTATACAAAGAACATTAAAAACTCCTAGCAAAACA |
| | | TTAAAAGGACGTTGAATAGTTGATTATTTTAAATAAGGGGCAAGC |
| | | TCTAAAATAAATTATTATTATAGTTGTAGTCCTGCGAGTATAAAG |
| | | TTTTATGCAATTTATGGAAAGAAATAAAACTTAAAGCAGAATAAC |
| | | CTGACAAGGTTATTGAAGAAACCTAATAGTTTCTTTGGCAATATT |
| | | AGTGAAAACGATCAAAATATATTGTAACTACAATATAAGAGATCG |
| | | TCGGTTATCCATATAATCGCGACAGACTGGGTCACTAATGGGTGG |
| | | CTGAAATGCTGCTTAATGCACAGTCGAAACTTTATATCAAGGCGA |
| | | TATATATCGCTCTAATATAGGTGATAAACGAACAATAAAGTTATC |
| | | ATCGCTTTTGTACTTTGCTACAAAAGTAAGTTTGTATGTTTTACC |
| | | TTATGGACAAATGTCATTATGAGGTGCTACAGTTATTACTAATTT |
| | | AATTAGTGCTGTTCCTTGAATTGGACAAGAGCCACCATGCCCGCC |
| | | ATGAAGATCGAGTGCCGCATCACCGGCACCCTGAACGGCGTGGAG |
| | | TTCGAGCTGGTGGGCGGCGGAGAGGGCACCCCCGAGCAGGGCCGC |
| | | ATGACCAACAAGATGAAGAGCACCAAAGGCGCCCTGACCTTCAGC |
| | | CCCTACCTGCTGAGCCACGTGATGGGCTACGGCTTCTACCACTTC |
| | | GGCACCTACCCCAGCGGCTACGAGAACCCCTTCCTGCACGCCATC |
| | | AACAACGGCGGCTACACCAACACCCGCATCGAGAAGTACGAGGAC |
| | | GGCGGCGTGCTGCACGTGAGCTTCAGCTACCGCTACGAGGCCGGC |
| | | CGCGTGATCGGCGACTTCAAGGTGGTGGGCACCGGCTTCCCCGAG |
| | | GACAGCGTGATCTTCACCGACAAGATCATCCGCAGCAACGCCACC |
| | | GTGGAGCACCTGCACCCCATGGGCGATAACGTGCTGGTGGGCAGC |
| | | TTCGCCCGCACCTTCAGCCTGCGCGACGGCGGCTACTACAGCTTC |
| | | GTGGTGGACAGCCACATGCACTTCAAGAGCGCCATCCACCCCAGC |
| | | ATCCTGCAGAACGGGGGCCCCATGTTCGCCTTCCGCCGCGTGGAG |
| | | GAGCTGCACAGCAACACCGAGCTGGGCATCGTGGAGTACCAGCAC |
| | | GCCTTCAAGACCCCCATCGCCTTCGCCAGATCTCGAGCTCGATGA |
| | | TAATTAAAACAGCCTGTGGGTTGATCCCACCCACAGGCCCATTGG |
| | | GCGCTAGCACTCTGGTATCACGGTACCTTTGTGCGCCTGTTTTAT |
| | | ACCCCCTCCCCCAACTGTAACTTAGAAGTAACACACACCGATCAA |
| | | CAGTCAGCGTGGCACACCAGCCACGTTTTGATCAAGCACTTCTGT |
| | | TACCCCGGACTGAGTATCAATAGACTGCTCACGCGGTTGAAGGAG |
| | | AAAGCGTTCGTTATCCGGCCAACTACTTCGAAAAACCTAGTAACA |
| | | CCGTGGAAGTTGCAGAGTGTTTCGCTCAGCACTACCCCAGTGTAG |
| | | ATCAGGTCGATGAGTCACCGCATTCCCCACGGGCGACCGTGGCGG |
| | | TGGCTGCGTTGGCGGCCTGCCCATGGGGAAACCCATGGGACGCTC |
| | | TAATACAGACATGGTGCGAAGAGTCTATTGAGCTAGTTGGTAGTC |
| | | CTCCGGCCCCTGAATGCGGCTAATCCTAACTGCGGAGCACACACC |
| | | CTCAAGCCAGAGGGCAGTGTGTCGTAACGGGCAACTCTGCAGCGG |
| | | AACCGACTACTTTGGGTGTCCGTGTTTCATTTTATTCCTATACTG |
| | | GCTGCTTATGGTGACAATTGAGAGATCGTTACCATATAGCTATTG |
| | | GATTGGCCATCCGGTGACTAATAGAGCTATTATATATCCCTTTGT |
| | | TGGGTTTATACCACTTAGCTTGAAAGAGGTTAAAACATTACAATT |
| | | CATTGTTAAGTTGAATACAGCAAACGCATCTTATAGAGCACCAAG |
| | | AACATTGACATGAGTTATAGGTACAATAATCCTTATCGTTATGAT |
| | | AGTTACAGGATTCCTGGGTTACCATACAATAGCCCAAAACGACTA |
| | | TAATAATAATAAAATAACAACTACAACAACCTTTAACGATAAAAG |
| | | ATATTATTCAACATCGAGAAATAATGAAGAGGAGACTTCGTTCTC |
| | | ACGTATAAATAAGTTTTTATTAGCCAA |
| Construct 3 | 16 | TAATACGACTCACTATAGGGTCATCCTTACATATTCAACGTAAAT |
| | | AATACTGTATATGGCGAATTTAATAGTATAGTTGAAGCAGCAGAA |
| | | GCTTTAAATTGTTCAACTAAAACTATACAAAGAACATTAAAAACT |
| | | CCTAGCAAAACATTAAAAGGACGTTGAATAGTTGATTATTTTAAA |
| | | TAAGGGGCAAGCTCTAAAATAAATTATTATTATAGTTGTAGTCCT |
| | | GCGAGTATAAAGTTTTATGCAATTTATGGAAAGAAATAAAACTTA |
| | | AAGCAGAATAACCTGACAAGGTTATTGAAGAAACCTAATAGTTTC |
| | | TTTGGCAATATTAGTGAAAACGATCAAAATATATTGTAACTACAA |
| | | TATAAGAGATCGTCGGTTATCCATATAATCGCGACAGACTGGGTC |
| | | ACTAATGGGTGGCTGAAATGCTGCTTAATGCACAGTCGAAACTTT |
| | | ATATCAAGGCGATATATATCGCTCTAATATAGGTGATAAACGAAC |
| | | AATAAAGTTATCATCGCTTTTGTACTTTGCTACAAAAGTAAGTTT |
| | | GTATGTTTTACCTTATGGACAAATGTCATTATGAGGTGCTACAGT |
| | | TATTACTAATTTAATTAGTGCTGTTCCTTGAATTGGACAAGAGCC |
| | | ACCATGCCCGCCATGAAGATCGAGTGCCGCATCACCGGCACCCTG |
| | | AACGGCGTGGAGTTCGAGCTGGTGGGCGGCGGAGAGGGCACCCCC |
| | | GAGCAGGGCCGCATGACCAACAAGATGAAGAGCACCAAAGGCGCC |
| | | CTGACCTTCAGCCCCTACCTGCTGAGCCACGTGATGGGCTACGGC |
| | | TTCTACCACTTCGGCACCTACCCCAGCGGCTACGAGAACCCCTTC |
| | | CTGCACGCCATCAACAACGGCGGCTACACCAACACCCGCATCGAG |
| | | AAGTACGAGGACGGCGGCGTGCTGCACGTGAGCTTCAGCTACCGC |
| | | TACGAGGCCGGCCGCGTGATCGGCGACTTCAAGGTGGTGGGCACC |
| | | GGCTTCCCCGAGGACAGCGTGATCTTCACCGACAAGATCATCCGC |
| | | AGCAACGCCACCGTGGAGCACCTGCACCCCATGGGCGATAACGTG |
| | | CTGGTGGGCAGCTTCGCCCGCACCTTCAGCCTGCGCGACGGCGGC |
| | | TACTACAGCTTCGTGGTGGACAGCCACATGCACTTCAAGAGCGCC |
| | | ATCCACCCCAGCATCCTGCAGAACGGGGGCCCCATGTTCGCCTTC |
| | | CGCCGCGTGGAGGAGCTGCACAGCAACACCGAGCTGGGCATCGTG |
| | | GAGTACCAGCACGCCTTCAAGACCCCCATCGCCTTCGCCAGATCT |
| | | CGAGCTCGATGATAATTAAAACAGCCTGTGGGTTGATCCCACCCA |
| | | CAGGCCCATTGGGCGCTAGCACTCTGGTATCACGGTACCTTTGTG |
| | | CGCCTGTTTTATACCCCCTCCCCCAACTGTAACTTAGAAGTAACA |
| | | CACACCGATCAACAGTCAGCGTGGCACACCAGCCACGTTTTGATC |
| | | AAGCACTTCTGTTACCCCGGACTGAGTATCAATAGACTGCTCACG |
| | | CGGTTGAAGGAGAAAGCGTTCGTTATCCGGCCAACTACTTCGAAA |
| | | AACCTAGTAACACCGTGGAAGTTGCAGAGTGTTTCGCTCAGCACT |
| | | ACCCCAGTGTAGATCAGGTCGATGAGTCACCGCATTCCCCACGGG |
| | | CGACCGTGGCGGTGGCTGCGTTGGCGGCCTGCCCATGGGGAAACC |
| | | CATGGGACGCTCTAATACAGACATGGTGCGAAGAGTCTATTGAGC |
| | | TAGTTGGTAGTCCTCCGGCCCCTGAATGCGGCTAATCCTAACTGC |
| | | GGAGCACACACCCTCAAGCCAGAGGGCAGTGTGTCGTAACGGGCA |
| | | ACTCTGCAGCGGAACCGACTACTTTGGGTGTCCGTGTTTCATTTT |
| | | ATTCCTATACTGGCTGCTTATGGTGACAATTGAGAGATCGTTACC |
| | | ATATAGCTATTGGATTGGCCATCCGGTGACTAATAGAGCTATTAT |
| | | ATATCCCTTTGTTGGGTTTATACCACTTAGCTTGAAAGAGGTTAA |
| | | AACATTACAATTCATTGTTAAGTTGAATACAGCAAACGCATCTTA |
| | | TAGAGCACCAAGAACATTGACATGAGTTATAGGTACAATAATCCT |
| | | TATCGTTATGATAGTTACAGGATTCCTGGGTTACCATACAATAGC |
| | | CCAAAACGACTATAATAATAATAAAATAACAACTACAACAACCTT |
| | | TAACGATAAAAGATATTATTCAACATCGAGAAATAATGAAGAGGA |
| | | GACTTCGTTCTCACGTATAAATAAGTTTTTATTAGCCAAAAATCT |
| | | TAATCCTGTTTTTATCTATCATAATCTAAATGAAGATTCAGTTCG |
| | | TAGAAATATAGCTAAAGAAACTAAGGGACTTAGTGGTATTTATAT |
| | | GATCTTAAATAAAGAAACTTTAAGTTATTATATTGGATCAGCTTC |
| | | TACTGACAGAATTAACTCTAGATTTTCAAAACATTTAATATATTT |
| | | AAATGGTAGTAAAATAGTTAAAAATTCAGTTAATAAATATGGTTT |
| | | ACATAATTTTGCCTTTATTGTATTAGAATTATTCCCTGAAATAGT |
| | | TAATCAAGAAAATAATAAAAAATTATTAGATTTAGAAGATTTTTA |
| | | TCTAAAATCTCTTTTACCTGACTATAATATATTAACCGAAGCAGG |
| | | ATCTAGCTTTGGATATAAACATACTGAAGTTAATAGAATAAAGAT |
| | | GAAAGCTAATTATAGTGAGAAGCGTAGAGAAGAAATAGGTAGTTT |
| | | GAATAGAGGTAAAACTTTATCTTCTGAAACTATAGAAACTATGAG |
| | | ACAATCAGCTTTAAATAGAAAACCTTTAGACTATACAGAACAAGG |
| | | TGTTTTAAATATGAAAAAGAATTCTAAGCCTATTATAGTAAAAGA |
| | | AT |
| Exon 1-2 | 17 | ATTCC |
| Exon 2-2 | 18 | TTACC |
| Exon 1-3 | 19 | ACAGGATTCC |
| Exon 2-3 | 20 | TTACCTTATG |
| Exon 1-4 | 21 | TATGATAGTTACAGGATTCC |
| Exon 2-4 | 22 | TTACCTTATGGACAAATGTC |
| Exon 1-5 | 23 | TCCTTATCGTTATGATAGTTACAGGATTCC |
| Exon 2-5 | 24 | TTACCTTATGGACAAATGTCATTATGAGGT |
| Exon 1-6 | 25 | GGTACAATAATCCTTATCGTTATGATAGTTACAGGATTCC |
| Exon 2-6 | 26 | TTACCTTATGGACAAATGTCATTATGAGGTGCTACAGTTA |
| Exon 1-7 | 27 | ATGAGTTATAGGTACAATAATCCTTATCGTTATGATAGTTACAGG |
| | | ATTCC |
| Exon 2-7 | 28 | TTACCTTATGGACAAATGTCATTATGAGGTGCTACAGTTATTACT |
| | | AATTT |
| F2-3′-intron-2 | 29 | AAATCTTAATCCTGTTTTTATCTATCATAATCTAAATGAAGATTC |
| | | AGTTCGTAGAAATATAGCTAAAGAAACTAAGGGACTTAGTGGTAT |
| | | TTATATGATCTTAAATAAAGAAACTTTAAGTTATTATATTGGATC |
| | | AGCTTCTACTGACAGAATTAACTCTAGATTTTCAAAACATTTAAT |
| | | ATATTTAAATGGTAGTAAAATAGTTAAAAATTCAGTTAATAAATA |
| | | TGGTTTACATAATTTTGCCTTTATTGTATTAGAATTATTCCCTGA |
| | | AATAGTTAATCAAGAAAATAATAAAAAATTATTAGATTTAGAAGA |
| | | TTTTTATCTAAAATCTCTTTTACCTGACTATAATATATTAACCGA |
| | | AGCAGGATCTAGCTTTGGATATAAACATACTGAAGTTAATAGAAT |
| | | AAAGATGAAAGCTAATTATAGTGAGAAGCGTAGAGAAGAAATAGG |
| | | TAGTTTGAATAGAGGTAAAACTTTATCTTCTGAAACTATAGAAAC |
| | | TATGAGACAATCAGCTTTAAATAGAAAACCTTTAGACTATACAGA |
| | | ACAAGGTGTTTTAAATATGAAAAAGAATTCTAAGCCTATTATAGT |
| | | AAAAGAATTAAATAATACTGTATATGGCGAATTTAATAGTATAGT |
| | | TGAAGCAGCAGAAGCTTTAAATTGTTCAACTAAAACTATACAAAG |
| | | AACATTAAAAACTCCTAGCAAAACATTAAAAGGACGTTGAATAGT |
| | | TGATTATTTTAAATAAGGGGCAAGCTCTAAAATAAATTATTATTA |
| | | TAGTTGTAGTCCTGCGAGTATAAAGTTTTATGCAATTTATGGAAA |
| | | GAAATAAAACTTAAAGCAGAATAACCTGACAAGGTTATTGAAGAA |
| | | ACCTAATAGTTTCTTTGGCAATATTAGTGAAAACGATCAAAATAT |
| | | ATTGTAACTACAATATAAGAGATCGTCGGTTATCCATATAATCGC |
| | | GACAGACTGGGTCACTAATGGGTGGCTGAAATGCTGCTTAATGCA |
| | | CAGTCGAAACTTTATATCAAGGCGATATATATCGCTCTAATATAG |
| | | GTGATAAACGAACAATAAAGTTATCATCGCTTTTGTACTTTGCTA |
| | | CAAAAGTAAGTTTG |
| F2-5′-intron-2 | 30 | CCATACAATAGCCCAAAACGACTATAATAATAATAAAATAACAAC |
| | | TACAACAACCTTTAACGATAAAAGATATTATTCAACATCGAGAAA |
| | | TAATGAAGAGGAGACTTCGTTCTCACGTATAAATAAGTTTTTATT |
| | | AGCCAA |
| F2-3′-intron-3 | 31 | AAATCTTAATCCTGTTTTTATCTATCATAATCTAAATGAAGATTC |
| | | AGTTCGTAGAAATATAGCTAAAGAAACTAAGGGACTTAGTGGTAT |
| | | TTATATGATCTTAAATAAAGAAACTTTAAGTTATTATATTGGATC |
| | | AGCTTCTACTGACAGAATTAACTCTAGATTTTCAAAACATTTAAT |
| | | ATATTTAAATGGTAGTAAAATAGTTAAAAATTCAGTTAATAAATA |
| | | TGGTTTACATAATTTTGCCTTTATTGTATTAGAATTATTCCCTGA |
| | | AATAGTTAATCAAGAAAATAATAAAAAATTATTAGATTTAGAAGA |
| | | TTTTTATCTAAAATCTCTTTTACCTGACTATAATATATTAACCGA |
| | | AGCAGGATCTAGCTTTGGATATAAACATACTGAAGTTAATAGAAT |
| | | AAAGATGAAAGCTAATTATAGTGAGAAGCGTAGAGAAGAAATAGG |
| | | TAGTTTGAATAGAGGTAAAACTTTATCTTCTGAAACTATAGAAAC |
| | | TATGAGACAATCAGCTTTAAATAGAAAACCTTTAGACTATACAGA |
| | | ACAAGGTGTTTTAAATATGAAAAAGAATTCTAAGCCTATTATAGT |
| | | AAAAGAATTAAATAATACTGTATATGGCGAATTTAATAGTATAGT |
| | | TGAAGCAGCAGAAGCTTTAAATTGTTCAACTAAAACTATACAAAG |
| | | AACATTAAAAACTCCTAGCAAAACATTAAAAGGACGTTGAATAGT |
| | | TGATTATTTTAAATAAGGGGCAAGCTCTAAAATAAATTATTATTA |
| | | TAGTTGTAGTCCTGCGAGTATAAAGTTTTATGCAATTTATGGAAA |
| | | GAAATAAAACTTAAAGCAGAATAACCTGACAAGGTTATTGAAGAA |
| | | ACCTAATAGTTTCTTTGGCAATATTAGTGAAAACGATCAAAATAT |
| | | ATTGTAACTACAATATAAGAGATCGTCGGTTATCCATATAATCGC |
| | | GACAGACTGGGTCACTAATGGGTGGCTGAAATGCTGCTTAATGCA |
| | | CAGTCGAAACTTTATATCAAGGCGATATATATCGCTCTAATATAG |
| | | GTGATAAACGAACAATAAAGTTATCATCGCTTTTGTACTTTGCTA |
| | | CAAAAGTAAGTTTGTGTT |
| F2-5′-intron-3 | 32 | TGGGTCCATACAATAGCCCAAAACGACTATAATAATAATAAAATA |
| | | ACAACTACAACAACCTTTAACGATAAAAGATATTATTCAACATCG |
| | | AGAAATAATGAAGAGGAGACTTCGTTCTCACGTATAAATAAGTTT |
| | | TTATTAGCCAA |
| F2-5′-intron-4 | 33 | TCCCTTACCATACAATAGGGGAAAACGACTATAATAATAATAAAA |
| | | TAACAACTACAACAACCTTTAACGATAAAAGATATTATTCAACAT |
| | | CGAGAAATAATGAAGAGGAGACTTCGTTCTCACGTATAAATAAGT |
| | | TTTTATTAGCCAA |
| F2-5′-intron-5 | 34 | TAAATTACCATACAATAGTTTAAAACGACTATAATAATAATAAAA |
| | | TAACAACTACAACAACCTTTAACGATAAAAGATATTATTCAACAT |
| | | CGAGAAATAATGAAGAGGAGACTTCGTTCTCACGTATAAATAAGT |
| | | TTTTATTAGCCAA |
| F2-5′-intron-6 | 35 | TTTTTTACCATACAATAGAAAAAAACGACTATAATAATAATAAAA |
| | | TAACAACTACAACAACCTTTAACGATAAAAGATATTATTCAACAT |
| | | CGAGAAATAATGAAGAGGAGACTTCGTTCTCACGTATAAATAAGT |
| | | TTTTATTAGCCAA |
| F1-3′-intron-2 | 36 | TTCGTTCTCACGTATAAATAAGTTTTTATTAGCCAAAAATCTTAA |
| | | TCCTGTTTTTATCTATCATAATCTAAATGAAGATTCAGTTCGTAG |
| | | AAATATAGCTAAAGAAACTAAGGGACTTAGTGGTATTTATATGAT |
| | | CTTAAATAAAGAAACTTTAAGTTATTATATTGGATCAGCTTCTAC |
| | | TGACAGAATTAACTCTAGATTTTCAAAACATTTAATATATTTAAA |
| | | TGGTAGTAAAATAGTTAAAAATTCAGTTAATAAATATGGTTTACA |
| | | TAATTTTGCCTTTATTGTATTAGAATTATTCCCTGAAATAGTTAA |
| | | TCAAGAAAATAATAAAAAATTATTAGATTTAGAAGATTTTTATCT |
| | | AAAATCTCTTTTACCTGACTATAATATATTAACCGAAGCAGGATC |
| | | TAGCTTTGGATATAAACATACTGAAGTTAATAGAATAAAGATGAA |
| | | AGCTAATTATAGTGAGAAGCGTAGAGAAGAAATAGGTAGTTTGAA |
| | | TAGAGGTAAAACTTTATCTTCTGAAACTATAGAAACTATGAGACA |
| | | ATCAGCTTTAAATAGAAAACCTTTAGACTATACAGAACAAGGTGT |
| | | TTTAAATATGAAAAAGAATTCTAAGCCTATTATAGTAAAAGAATT |
| | | AAATAATACTGTATATGGCGAATTTAATAGTATAGTTGAAGCAGC |
| | | AGAAGCTTTAAATTGTTCAACTAAAACTATACAAAGAACATTAAA |
| | | AACTCCTAGCAAAACATTAAAAGGACGTTGAATAGTTGATTATTT |
| | | TAAATAAGGGGCAAGCTCTAAAATAAATTATTATTATAGTTGTAG |
| | | TCCTGCGAGTATAAAGTTTTATGCAATTTATGGAAAGAAATAAAA |
| | | CTTAAAGCAGAATAACCTGACAAGGTTATTGAAGAAACCTAATAG |
| | | TTTCTTTGGCAATATTAGTGAAAACGATCAAAATATATTGTAACT |
| | | ACAATATAAGAGATCGTCGGTTATCCATATAATCGCGACAGACTG |
| | | GGTCACTAATGGGTGGCTGAAATGCTGCTTAATGCACAGTCGAAA |
| | | CTTTATATCAAGGCGATATATATCGCTCTAATATAGGTGATAAAC |
| | | GAACAATAAAGTTATCATCGCTTTTGTACTTTGCTACAAAAGTAA |
| | | GTTTG |
| F3-3′-intron-2 | 37 | TAAATAATACTGTATATGGCGAATTTAATAGTATAGTTGAAGCAG |
| | | CAGAAGCTTTAAATTGTTCAACTAAAACTATACAAAGAACATTAA |
| | | AAACTCCTAGCAAAACATTAAAAGGACGTTGAATAGTTGATTATT |
| | | TTAAATAAGGGGCAAGCTCTAAAATAAATTATTATTATAGTTGTA |
| | | GTCCTGCGAGTATAAAGTTTTATGCAATTTATGGAAAGAAATAAA |
| | | ACTTAAAGCAGAATAACCTGACAAGGTTATTGAAGAAACCTAATA |
| | | GTTTCTTTGGCAATATTAGTGAAAACGATCAAAATATATTGTAAC |
| | | TACAATATAAGAGATCGTCGGTTATCCATATAATCGCGACAGACT |
| | | GGGTCACTAATGGGTGGCTGAAATGCTGCTTAATGCACAGTCGAA |
| | | ACTTTATATCAAGGCGATATATATCGCTCTAATATAGGTGATAAA |
| | | CGAACAATAAAGTTATCATCGCTTTTGTACTTTGCTACAAAAGTA |
| | | AGTTTG |
| Partial P1 | 38 | TGGGTTACCATACAATAGCCCA |
| domain | | |

Based on in-depth and extensive studies on circRNA synthesis in vitro, the inventors of the present disclosure found that group I intron of the cob gene of Fusarium oxysporum presents notable characteristics for catalyzing in vitro RNA circularization. Following re-organizing and modifications of the group I intron, gene of interest could be circularized from the linearized construct claimed by the present disclosure. In summary, when the construct is applied for circRNA synthesis in vitro, circRNA could be achieved carrying only 5 nt extraneous sequence, which is likely to result in low immunogenicity.

In the present disclosure, the Gene ID number of the cob gene of Fusarium oxysporum is LT906358, and the specific nucleotide sequence is as shown in SEQ ID NO: 1; among them, the nucleotide sequence of the group I intron contained in the cob gene is as shown in SEQ ID NO: 2.

In the present disclosure, the construct, following the 5′-3′ direction, includes 3′-intron, exon 2, gene of interest, exon 1, and 5′-intron. More specifically, the 3′-intron and the 5′-intron are formed by splitting the group I intron. And the splitting sites on the group I intron are located at positions 18-1020 of SEQ ID NO: 2, such as positions 18, 25, 108, 114, 737, 1000, 1020 or any sites therebetween, and the 5′-intron contains a P1 domain, and the P1 domain includes a nucleotide fragment having a sequence as shown in SEQ ID NO: 38.

In some specific embodiments, the splitting site is preferably located at position 144 of the sequence shown in SEQ ID NO: 2, namely, the nucleotide sequence of the 3′-intron in the construct is as shown in SEQ ID NO: 5, and the nucleotide sequence of the 5′-intron is as shown in SEQ ID NO: 6. In general, the obtained 3′-intron and 5′-intron are applied to the construct for in vitro RNA circularization, which has a relatively superior in vitro circularization efficiency and achieve circRNA in vitro.

In the present disclosure, the exon 1 is an exon fragment adjacent to the 5′ end of the group I intron, and its length is specifically 0-393 nt, such as 0 nt, 5 nt, 50 nt, 218 nt, 253 nt, 350 nt, 393 nt or any integer value therebetween; the exon 2 is an exon fragment adjacent to the 3′ end of the group I intron, and its length is specifically 0-780 nt, such as 0 nt, 58 nt, 93 nt, 218 nt, 324 nt, 630 nt, 736 nt, 748 nt, 780 nt or any integer value therebetween. In general, an introduction of the exon 1 and exon 2 into the construct is to maintain or improve the self-cleaving catalytic activity of the group I intron.

In some specific embodiments, the lengths of exon 1 and exon 2 in the construct are preferably 0 nt, namely, no additional exon fragment adjacent to the group I intron being introduced into the construct. In general, the group I intron on the cob gene of Fusarium oxysporum remains self-cleaving catalytic activity. Even without introducing exon fragments, it still maintains a certain self-cleaving catalytic activity, and addresses the issue of long exogenous sequences residues in the existing in vitro RNA circularization.

In the present disclosure, P10 domain is preferably excluded in the 3′-intron, and the specific nucleotide sequence is as shown in SEQ ID NO: 29. In general, although the deletion of the P10 domain in the 3′-intron reduces the self-cleaving catalytic activity of ribozymes, more importantly, it achieves an excellent effect of reducing residual exogenous sequences in circular RNA.

In some specific embodiments, the nucleotide sequence of the preferred 3′-intron in the construct is as shown in SEQ ID NO: 29, and the nucleotide sequence of the 5′-intron is as shown in SEQ ID NO: 6. In general, the use of the construct can achieve in vitro RNA circularization, and only 5 nt of exogenous sequences remain in the obtained circular RNA, with low immunogenicity.

In the present disclosure, the construct preferably further includes a promoter located at the 5′ end of the 3′-intron, and the promoter serves as an important initiation element for in vitro transcription. In some specific embodiments, specific examples of the promoter include but are not limited to: one or more of T7 promoter, T3 promoter and SP6 promoter. In some preferred embodiments, the construct includes a T7 promoter located at the 5′ end of the 3′-intron, and the T7 promoter is derived from a T7 bacteriophage to initiate transcription of the construct, and its specific nucleotide sequence is as shown in SEQ ID NO: 11.

In the present disclosure, the construct preferably further includes an IRES sequence and/or an IRES-like sequence located between the gene of interest and the exon 1. The IRES sequence and/or IRES-like sequence are used as internal ribosome entry sites to initiate the translation of circular RNA in vivo. More specifically, specific examples of the IRES sequence and/or IRES-like sequence include but are not limited to: one or more of CVB3 IRES sequence, EMCV IRES sequence, and EV29 IRES sequence.

In some specific embodiments, the IRES sequence is preferably a CVB3 IRES sequence, and its specific nucleotide sequence is as shown in SEQ ID NO: 13. In general, the circular RNA obtained by in vitro circularization of the construct has high translation efficiency and can achieve efficient and specific gene expression.

In the present disclosure, the construct can be specifically DNA and/or RNA. When the construct is DNA, it can be loaded onto a plasmid vector, and the linear RNA obtained after the transcription of the construct will be subject to an in vitro circularization reaction to obtain circular RNA. When the construct is RNA, it can be directly used as a raw material for in vitro circularization reaction to obtain circular RNA. In some specific embodiments, the construct is preferably DNA. In general, the construct has higher stability.

The present disclosure further provides a method for constructing a construct for in vitro RNA circularization. The construction method is based on tapping the potential of the group I intron of the cob gene of Fusarium oxysporum to design and induce circRNA synthesis in vitro and gene of interest could be internalized in circRNA.

In the present disclosure, the construction method specifically includes: performing a structural analysis on the group I intron of the cob gene of Fusarium oxysporum, obtaining splitting sites related to the enzyme activity of the group I intron, and making the construct framework; and introducing the gene of interest based on the construct framework, and completing the construct.

In the present disclosure, the construct preferably further includes a functionalized sequence; specific examples of the functionalized sequence include but are not limited to: promoter and/or IRES sequence. Among them, the promoter and IRES sequence can be but are not limited to the promoter and IRES sequence described above, which are a type of functionalized sequences commonly used. Those skilled in the art can select suitable sequences according to their needs, and the present disclosure does not particularly limit them.

The present disclosure further provides a construct obtained by the preceding construction method, the construct, following the 5′-3′ direction, includes 3′-intron, exon 2, gene of interest, exon 1, and 5′-intron, and the construct can be partially identical or completely identical to the construct described above.

The present disclosure provides a method for RNA circularization and the construct used in the method is DNA, and specifically the method includes: taking a construct for transcription to obtain a linear RNA molecule; and inducing the linear RNA molecule for circularization to obtain the circular RNA.

In the present disclosure, the transcription preferably indicates in vitro transcription, specifically refers to a process of generating RNA by catalytic reaction of RNA polymerase independence of the cell system, using DNA as a construct and four ribonucleotides A/G/C/U as raw materials in an appropriate buffer system, imitating the reaction process in vivo. The reagents and conditions used in the in vitro transcription are conventional technical means in this field, limited to achieving in vitro circularization of linear RNA, and the present disclosure does not particularly limit it.

In the present disclosure, the circularization is achieved based on the self-cleaving catalytic activity of the 3′-intron and 5′-intron in the construct. The reagents and conditions used in the circularization are conventional technical means in this field, limited to achieving in vitro circularization of linear RNA, and the present disclosure does not particularly limit it.

The present disclosure further provides a circular RNA, and the circular RNA is prepared by the preceding method for RNA circularization. The circular RNA contains only extremely low exogenous sequence (5 nt) and has low immunogenicity. When the circular RNA is introduced into an organism, it can achieve efficient expression of the target protein without triggering a serious immune response in the body, and has high biosafety.

The present disclosure further provides applications of the construct and/or circular RNA described above in the preparation of medicines, vaccines and/or cells highly expressing target genes.

In the present disclosure, the circular RNA obtained by in vitro circularization of the preceding construct and/or the preceding circular RNA contains only a small amount of exogenous sequence (5 nt), which has low immunogenicity. When the circular RNA is introduced into an organism, it can achieve efficient expression of the target protein without triggering serious immune responses in the organism, and has high biosafety. Therefore, it can be well applied to the preparation of medicines, vaccines and/or cells to increase expressions.

The embodiments of the present disclosure are described in detail below, and the examples of the embodiments are intended to explain the present disclosure and should not be comprehended as confining the present disclosure. If the specific technology or conditions are not specified in the embodiments, it shall be carried out according to the technology or conditions described in the literature in this field or in accordance with the product manual. The reagents or instruments used without indicating the manufacturer are all conventional products that can be obtained through commercial purchase.

### Embodiment 1

This embodiment is used to illustrate a construct and a construction method thereof.

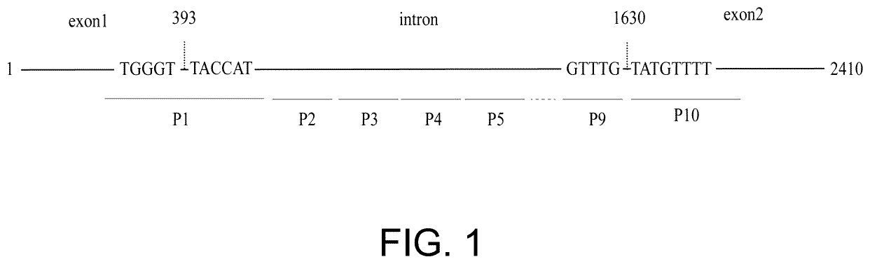

The construct is obtained by analyzing and designing the group I intron on the cob gene (Gene ID: LT906358, SEQ ID NO: 1) of Fusarium oxysporum as shown in FIG. 1. Its construction method specifically includes the following:

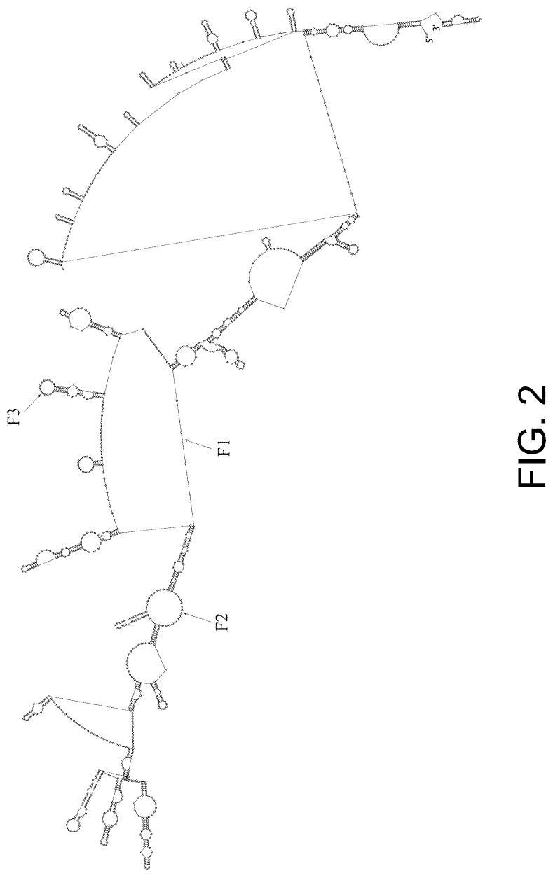

1. A secondary structure of the group I intron (the nucleotide sequence thereof is as shown in SEQ ID NO: 2) is analyzed, and multiple splitting sites and adjacent exon fragments highly correlated with the self-cleaving catalytic activity of the group I intron are optimized. The secondary structure of the group I intron and the splitting sites thereon are specifically shown in FIG. 2; and
sequence permutation is performed based on the splitting sites and the adjacent exon fragments to obtain 3′-intron, initial exon 2, 5′-intron, initial exon 1, and, namely corresponding construct 1-3 (the nucleotide sequences thereof are as shown in SEQ ID NO: 11-13), as shown in Table 2.

TABLE 2

| | Position | 3′-intron | 5′-intron | |
|---|---|---|---|---|
| Splitting | (Refer to SEQ ID | Nucleotide | Nucleotide | |
| site | NO: 2) | sequence | sequence | Construct framework |
| F1 | 108 | SEQ ID NO: 3 | SEQ ID NO: 4 | Construct framework 1 |
| F2 | 144 | SEQ ID NO: 5 | SEQ ID NO: 6 | Construct framework 2 |
| F3 | 737 | SEQ ID NO: 7 | SEQ ID NO: 8 | Construct framework 3 |

| Adjacent exon | | |
|---|---|---|
| fragment | Position | Nucleotide sequence |
| initial exon 1 | 5′ end | SEQ ID NO: 9 |
| initial exon 2 | 3′ end | SEQ ID NO: 10 |

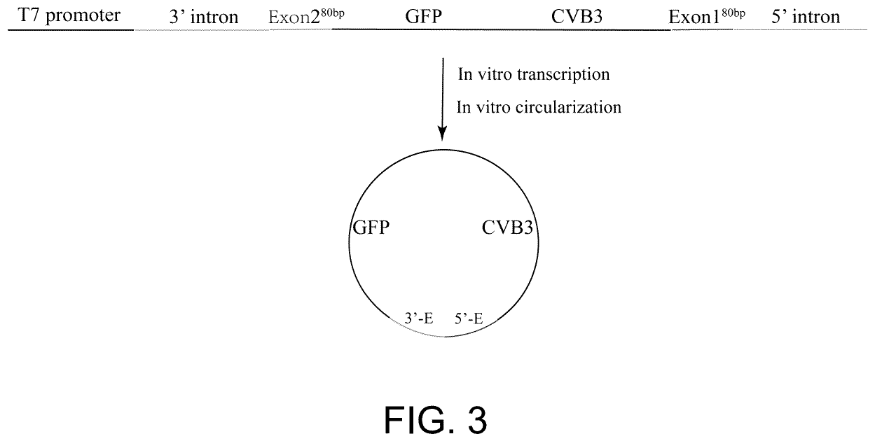

2. Referring to FIG. 3, a T7 promoter (the nucleotide sequence thereof is as shown in SEQ ID NO: 11), a gene of interest (GFP gene, the nucleotide sequence thereof is as shown in SEQ ID NO: 12) and a CVB3 IRES sequence (the nucleotide sequence thereof is shown in SEQ ID NO: 13) are systematically introduced into the corresponding positions in the construct frameworks 1-3 to construct constructs 1-3 (the nucleotide sequences thereof are as shown in SEQ ID NO: 14-16); and the constructs 1-3 obtained are commissioned to General Biol co. for synthesis.

3. (1) Plasmid construction: Different DNA elements were assembled using Gibson cloning kit (vazyme, catalog number: C112-02) and cloned into pUC57-KanR backbone to achieve constructs 1-3.

(2) In vitro transcription: IVT was performed following the manual of HiScribe® T7 high-efficiency RNA synthesis kit (NEB, catalog number: E2040S) and following the instructions to perform in vitro transcription on constructs 1-3 to obtain in vitro transcription products; and

Dnase I enzyme was further used to digest DNA constructs in the in vitro transcription products. LiCl solution with a concentration of 3M was applied for precipitation, and purification to obtain linear RNAs 1-3.

(3) In vitro circularization: Adjusting concentration of linear RNA with a final concentration of 50 nM, i.g., linear RNAs 1-3 and mixing them with the circularization buffer (including 20 mM Tris-HCl, 5 mM MgCl₂ and 25 mM NaCl), and adding GTP until the concentration of GTP in the solution is 2 mM, and performing in vitro circularization at 55° C. for 15 min to obtain circRNA 1-3;
purifying the circularization product using an RNA purification kit (NEB, catalog number T2040L). Following the manufacturer instructions, purified circularization products 1-3 are obtained;

The purified circularization product is digested with Rnase-R (Hzymes, catalog number: HBP004600-1) at 37° C. for 1 hour to obtain circular RNAs 1-3. The designs of circular RNAs 1-3 are shown in FIG. 3.

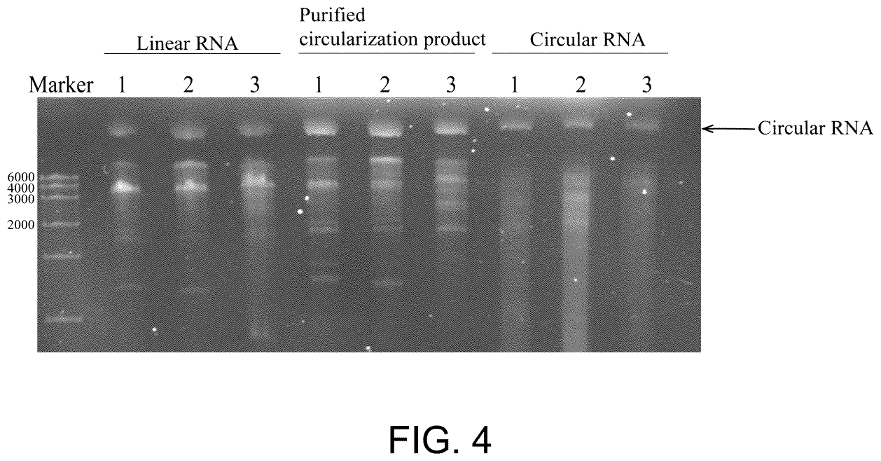

4. Perform 4% urea-PAGE denaturing gel electrophoresis using samples from individual steps, including linear RNAs 1-3, purified circularization products 1-3 and circular RNAs 1-3 obtained in “3”, and the amount of sample added and the conditions during the electrophoresis are the same. The circularization efficiency (%) of constructs 1-3 is calculated according to the following equation. The results are shown in FIG. 4 and Table 3.

$$Circularization efficiency ( \% ) = {X}_{1} / {X}_{2} \times 100 \%$$

Where X₁ is the band strength of a circular RNA in the gel electrophoresis diagram; and X₂ is the band strength of a linear RNA in the gel electrophoresis diagram.

TABLE 3

| | | Intensity of circular RNA band | Circularization |
|---|---|---|---|
| Construct | Splitting site | (Normalized to construct 2 intensity, %) | efficiency (%) |
| Construct 1 | F1 | 96 | 48.8 |
| Construct 2 | F2 | 100 | 48.1 |
| Construct 3 | F3 | 83 | 46.5 |

As shown in FIG. 4 and Table 3, the circularization efficiency of constructs 1-3 showed high circularization efficiency with more than 45%, and the intensity of the circular band in construct 2 is slightly higher than those of constructs 1 and 3.

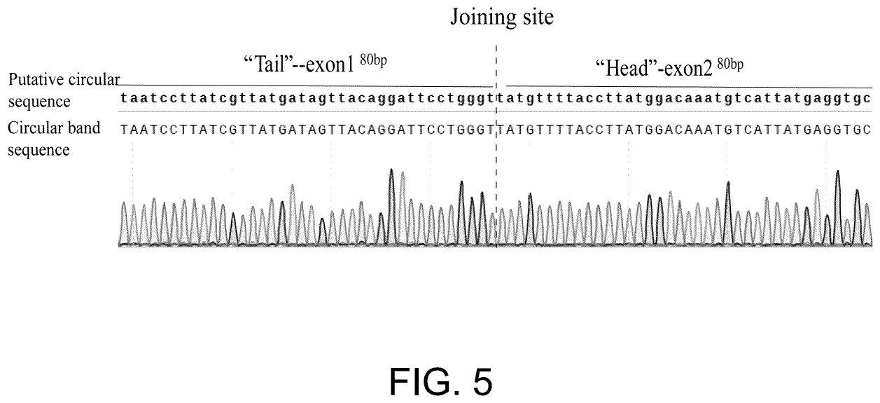

Cut and recover the circular band in construct 2, and perform reverse transcription, PCR and Sanger sequencing. The results are shown in FIG. 5. As seen from FIG. 5, the circular RNA 2 molecules are ligated head to end, further proving that it is a circular RNA product.

### Embodiment 2

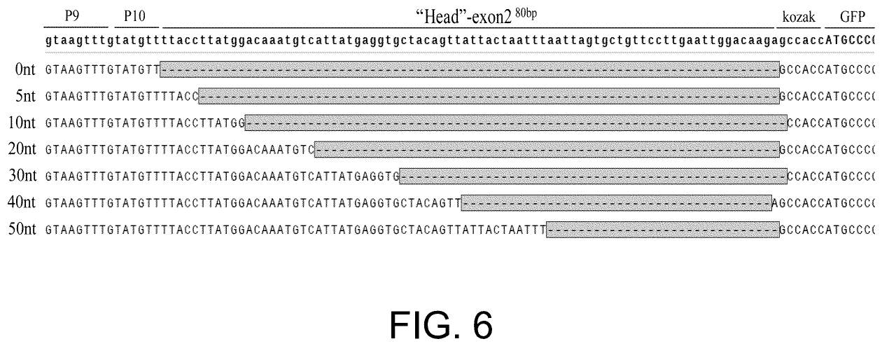

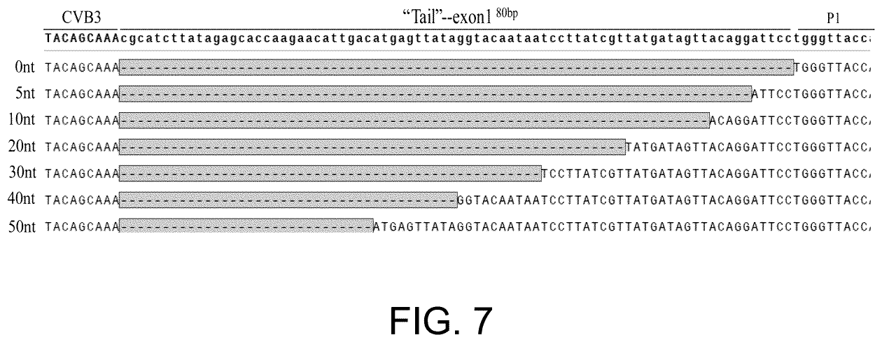

This embodiment is used to illustrate an optimized construct and a construction method thereof. The construct is based on the construct 2 synthesized in Embodiment 1, and are subject to stepwise nucleotide deletions from the initial exons 1 and 2. Referring to the construction method provided in Embodiment 1, constructs 4-10 are synthesized, and the obtained constructs 4-10 are obtained by homologous PCR amplification, as shown in FIG. 6, FIG. 7, and Table 4.

TABLE 4

| | | Nucleotide length (nt) | SEQ ID NO: |
|---|---|---|---|
| Construct 4 | Exon 1-1 | 0 | — |
| | Exon 2-1 | 0 | — |
| Construct 5 | Exon 1-2 | 5 | 17 |
| | Exon 2-2 | 5 | 18 |
| Construct 6 | Exon 1-3 | 10 | 19 |
| | Exon 2-3 | 10 | 20 |
| Construct 7 | Exon 1-4 | 20 | 21 |
| | Exon 2-4 | 20 | 22 |
| Construct 8 | Exon 1-5 | 30 | 23 |
| | Exon 2-5 | 30 | 24 |
| Construct 9 | Exon 1-6 | 40 | 25 |
| | Exon 2-6 | 40 | 26 |
| Construct 10 | Exon 1-7 | 50 | 27 |
| | Exon 2-7 | 50 | 28 |

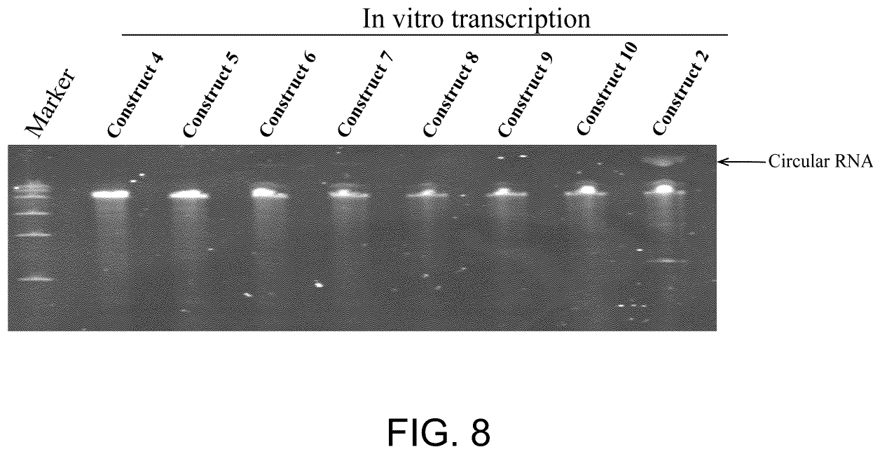

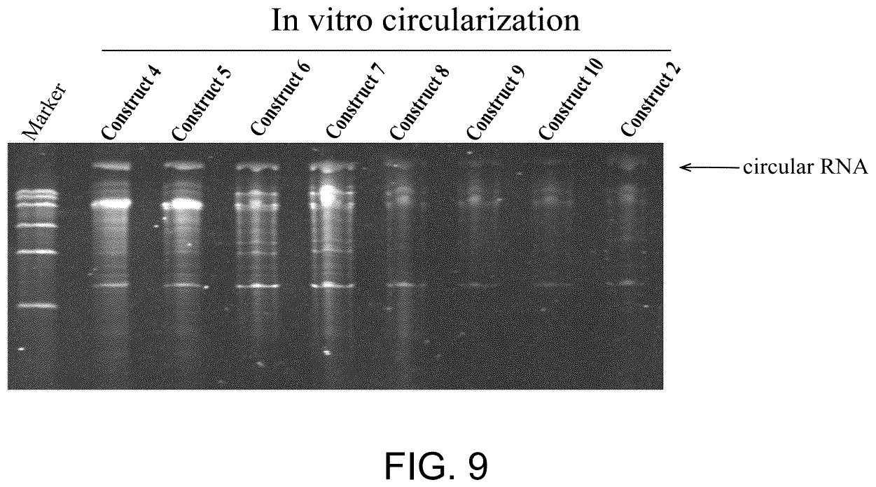

Referring to the method provided in Embodiment 1, perform in vitro transcription and in vitro circularization on constructs 4-10 to obtain linear RNAs 4-10 and circular RNAs 4-10; and the preceding linear RNAs 4-10 and circular RNAs 4-10 are subject to 4% urea-PAGE gel electrophoresis, and the results are shown in FIG. 8 and FIG. 9.

From the results shown in FIG. 8 and FIG. 9, it can be seen when construct 4 does not contain additional exon fragments, the in vitro transcription basically does not produce any visible circular RNA bands, but after being induced by circularization conditions, the circRNA could be well achieved even without introducing additional exons (namely, the exon length is 0 nt).

### Embodiment 3

This embodiment is used to illustrate a construct and a construction method thereof. The constructs 11-16 provided in this embodiment are obtained by modifying 3′-intron and 5′-intron with reference to the construction method provided in Embodiment 1, specifically including:

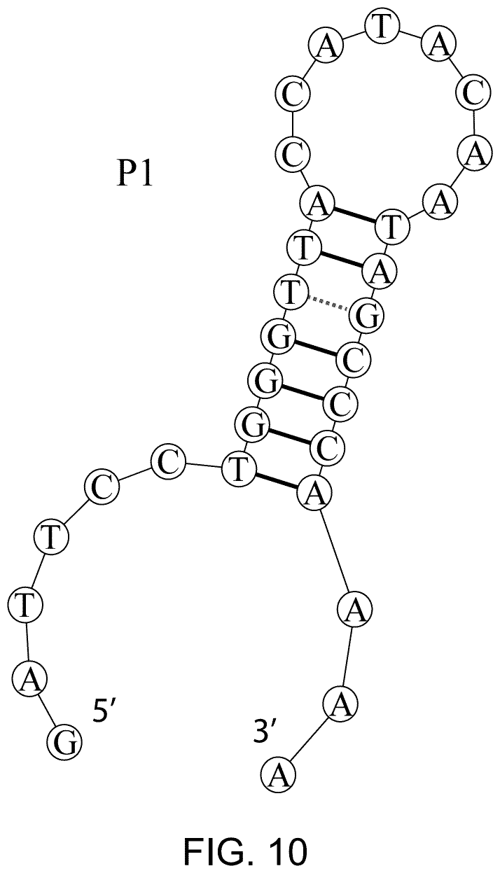

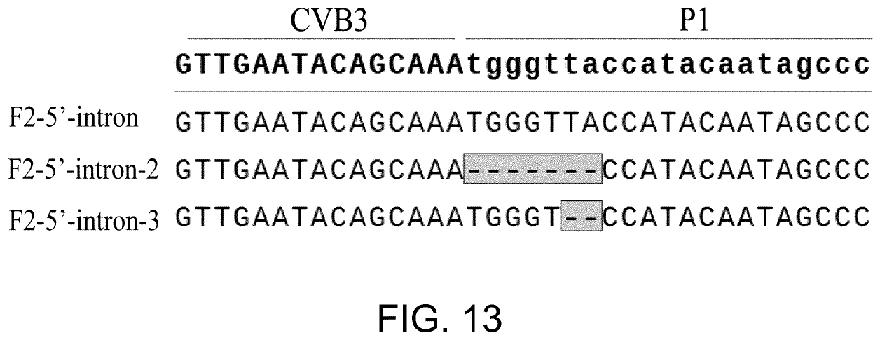

1. Construction of constructs 11-13: (1) Based on the construct 4 formed in Embodiment 2, modifying P10 and P1 domain in 3′-intron and 5′-intron, respectively, with reference to Table 5 and FIGS. 10-13 to obtain constructs 11-13 by homologous PCR amplification.

TABLE 5

| Group | Intron modification | Intron | SEQ ID NO: |
|---|---|---|---|
| Construct 11 | Delete the P10 domain of the 3′-intron | F2-3′-intron-2 | 29 |
| | | F2-5′-intron | 6 |
| Construct 12 | Simultaneously delete the P1 domain of the 5′- | F2-3′-intron-2 | 29 |
| | intron and the P10 domain of the 3′-intron | F2-5′-intron-2 | 30 |
| Construct 13 | Simultaneously delete TA bases in the P1 domain | F2-3′-intron-3 | 31 |
| | of the 5′-intron and the P10 domain of the 3′-intron | F2-5′-intron-3 | 32 |

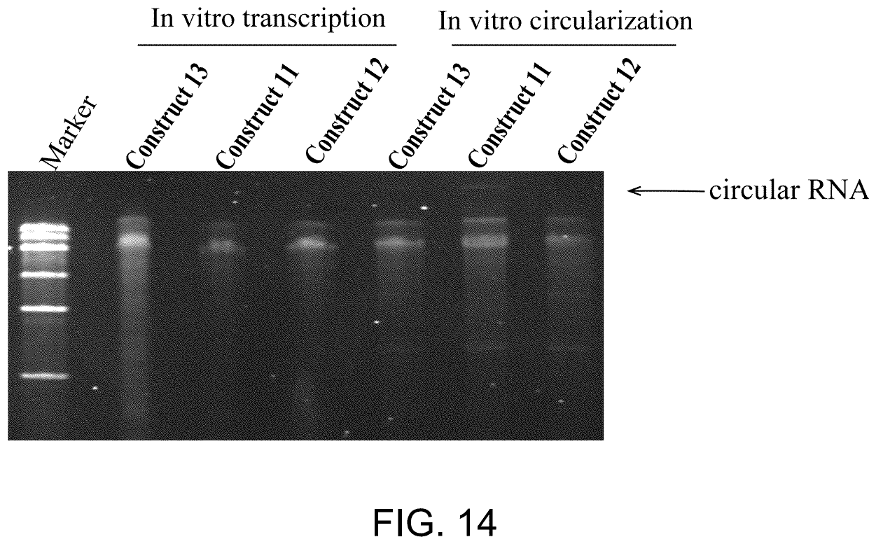

Referring to the method provided in Embodiment 1, perform in vitro transcription and in vitro circularization on constructs 11-13 to obtain linear RNAs 11-13 and circular RNAs 11-13; and the preceding purified circularization products 11-13 and circular RNAs 11-13 are subject to 4% urea-PAGE gel electrophoresis, and the amount of sample added and the conditions during the electrophoresis process are the same. The circularization efficiency (%) is obtained based on consistent calculation. The results are shown in FIG. 14 and Table 6.

TABLE 6

| | | Circularization |
|---|---|---|
| Construct | Intron | efficiency (%) |
| Construct 11 | F2-3′-intron-2 and F2-5′-intron | 6.0 |
| Construct 12 | F2-3′-intron-2 and F2-5′-intron-2 | — |
| Construct 13 | F2-3′-intron-3 and F2-5′-intron-3 | — |

From the results shown in FIG. 14 and Table 6, it can be seen that constructs 12 and 13 failed to produce circular RNA, while construct 11 achieved circRNA after being induced by circularization conditions even when the P10 domain is deleted, thereby obtaining circular RNA 11.

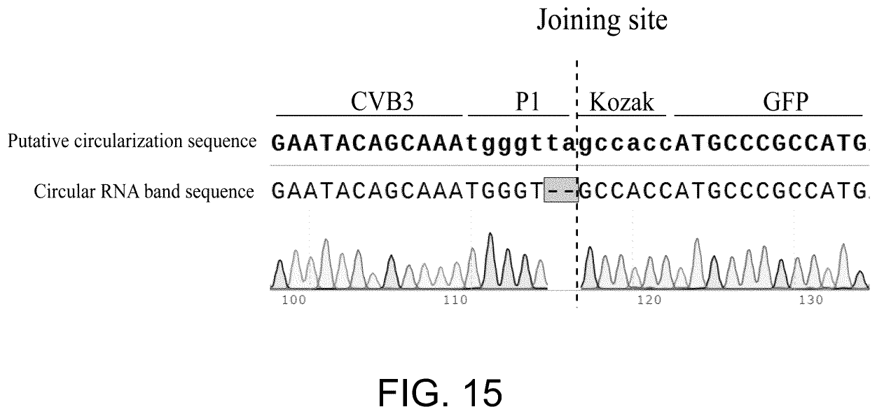

(2) Cut and recover the circular RNA 11 (obtained by subjecting the construct 11 to an in vitro circularization reaction), and perform reverse transcription, PCR and Sanger sequencing. The results are shown in FIG. 15.

From the results shown in FIG. 15, it can be seen that only 5 nucleotides (TGGGT) derived from the F2-3′-intron P1 domain remain in the circular RNA of construct 11, which can effectively address the issue of large immune response caused by the introduction of large fragments of exogenous sequences in the existing in vitro synthesis of circular RNA, and has good application prospects.

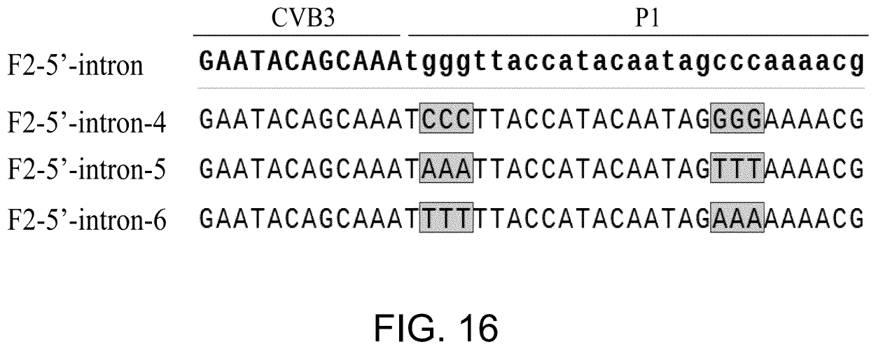

2. Construction of constructs 14-16: Based on the construct 11 obtained by the above construction, modify the P1 domain of 5′-intron with reference to Table 7 and FIG. 16 to obtain constructs 14-16 by homologous PCR amplification.

TABLE 7

| | Intron modification | Intron | SEQ ID NO: |
|---|---|---|---|
| Construct | Mutate consecutive GGG in the 5′-intron P1 domain | F2-5′-intron-4 | 33 |
| 14 | to CCC, and consecutive CCC in the complementary | | |
| | sequence to GGG | | |
| Construct | Mutate consecutive GGG in the 5′-intron P1 domain | F2-5′-intron-5 | 34 |
| 15 | to AAA, and consecutive CCC in the complementary | | |
| | sequence to TTT | | |
| Construct | Mutate consecutive GGG in the 5′-intron P1 domain | F2-5′-intron-6 | 35 |
| 16 | to TTT, and consecutive CCC in the complementary | | |
| | sequence to AAA | | |

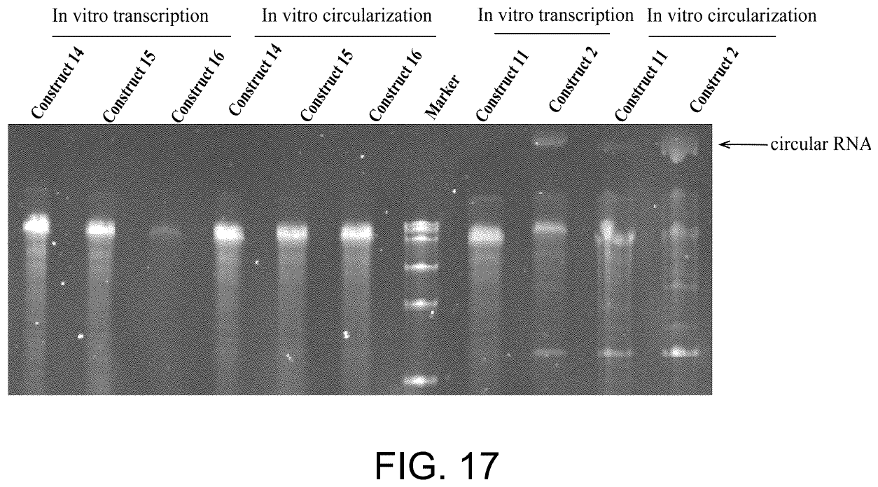

Referring to the method provided in Embodiment 1, in vitro transcription and in vitro circularization on constructs 14-16 were conducted to obtain linear RNAs 14-16, and were further purified and induced to form circular RNAs 14-16; and the purified precursor RNA products 14-16 and circular RNAs 14-16 are subject to 4% urea-PAGE gel electrophoresis, and the amount of sample added and the conditions during the electrophoresis process are the same. The circularization efficiency (%) is calculated based on above equation. The results are shown in FIG. 17 and Table 8.

TABLE 8

| | | Circularization |
|---|---|---|
| Construct | Intron | efficiency (%) |
| Construct 14 | F2-3′-intron-2 and F2-5′-intron-4 | — |
| Construct 15 | F2-3′-intron-2 and F2-5′-intron-5 | — |
| Construct 16 | F2-3′-intron-3 and F2-5′-intron-6 | — |

From the results shown in FIG. 17 and Table 8, it can be seen that constructs 14-16 were unable to generate circular RNA, suggesting that the P1 domain is necessary and unchangeable sequence for catalyzing the in vitro RNA circularization of the group I intron.

### Embodiment 4

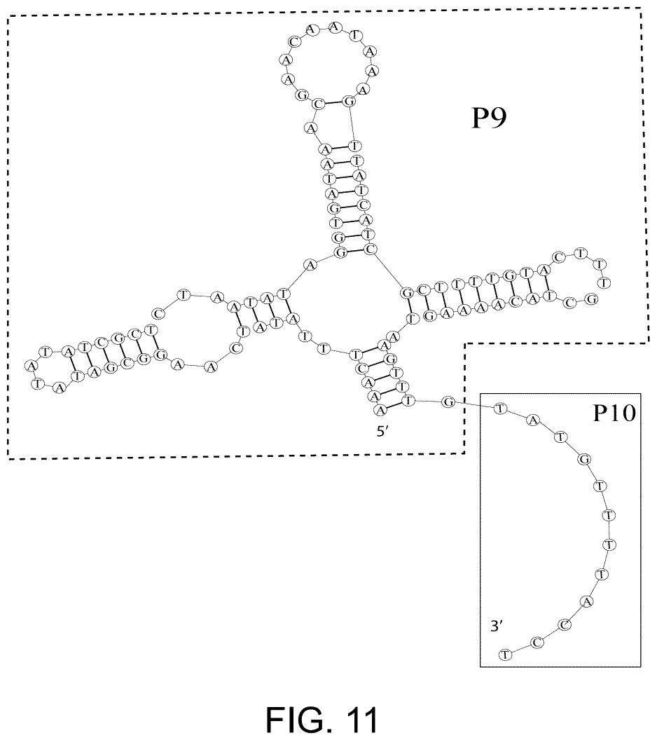

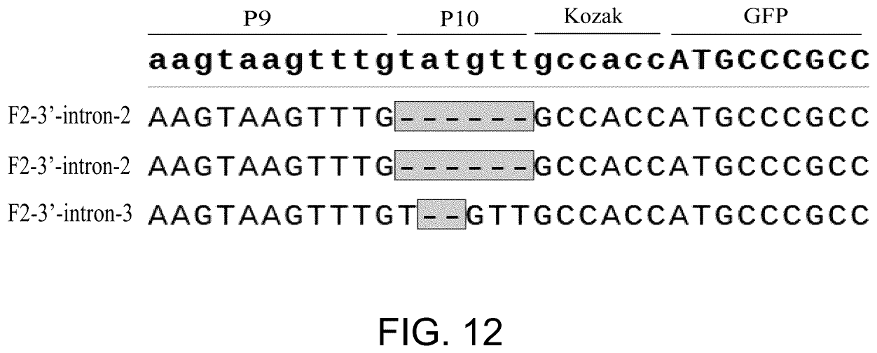

This embodiment is used to illustrate a construct and a construction method thereof. The construct is based on the construct 11 constructed in Embodiment 3. Modify the 3′-intron and 5′-intron as in constructs 1 and 3 (FIG. 11 and FIG. 12) with reference to the construction method provided in Embodiment 3 to obtain constructs 17 and 18, and the constructs 17 and 18 are obtained by homologous PCR amplification, as shown in Table 9.

TABLE 9

| | Original | | | |
|---|---|---|---|---|
| Construct | construct | Nucleotide modification | Intron | SEQ ID NO: |
| Construct | Construct 1 | (1) Delete the P10 domain of the | F1-3′-intron-2 | 36 |
| 17 | | 3′-intron; | | |
| | | (2) Without exon 1 and exon 2. | F1-5′-intron | 4 |
| Construct | Construct 3 | (1) Delete the P10 domain of the | F3-3′-intron-2 | 37 |
| 18 | | 3′-intron; | F3-5′-intron | 8 |
| | | (2) Without exon 1 and exon 2. | | |

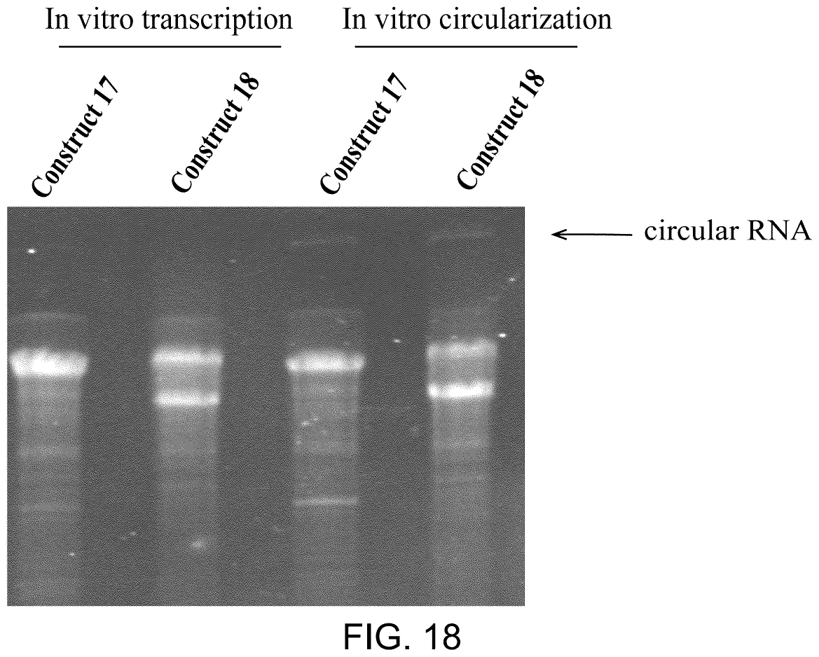

Referring to the method provided in Embodiment 1, perform in vitro transcription and in vitro circularization on constructs 17 and 18 to obtain linear RNAs 17 and 18 and circular RNAs 17 and 18; and the preceding purified linear RNA products 17 and 18 and circular RNAs 17 and 18 are subject to 4% urea-PAGE gel electrophoresis, and the amount of sample added and the conditions during the electrophoresis process are the same. Circularization efficiency (%) is obtained based on consistent calculation. The results are shown in FIG. 18 and Table 10.

TABLE 10

| | | Circularization |
|---|---|---|
| Construct | Intron | efficiency (%) |
| Construct 17 | F1-3′-intron-2 and F1-5′-intron | 2.6 |
| Construct 18 | F3-3′-intron-2 and F3-5′-intron | 2.3 |

From the results shown in FIG. 18 and Table 10, it can be seen that although the circularization efficiency of constructs 17 and 18 was not as good as that of construct 11, circular RNA could still be obtained, suggesting the versatile designs of low extraneous nucleotides in circRNA.

Although the embodiments of the present disclosure have been shown and described above, it can be comprehended that the above embodiments are exemplary and cannot be construed as confining the present disclosure. Scientists and technicians in this field may make changes, modifications, replacements and variations to the above embodiments within the scope of the present disclosure without departing from the principles and purposes of the present disclosure.

What is claimed is:

1. A construct for in vitro RNA synthesis and circularization, wherein the construct, following a direction of 5′-3′, comprises 3′-intron, exon 2, a gene of interest, exon 1, and 5′-intron;
wherein the 3′-intron and the 5′-intron are derived from a group I intron of a cob gene of Fusarium oxysporum with a nucleotide sequence as shown in SEQ ID NO: 1, and the 5′-intron comprises a P1 domain, and the P1 domain comprises a nucleotide fragment with a nucleotide sequence as shown in SEQ ID NO: 38; the exon 1 is an exon fragment adjacent to a 5′ end of the group I intron, and a length of the exon 1 is 0-393 nt; the exon 2 is an exon fragment adjacent to a 3′ end of the group I intron, and a length of the exon 2 is 0-780 nt.

2. The construct for in vitro RNA synthesis and circularization according to claim 1, wherein a nucleotide sequence of the group I intron is shown as in SEQ ID NO: 2.

3. The construct for in vitro RNA synthesis and circularization according to claim 1, wherein the 5′-intron and 3′-intron are obtained by splitting the group I intron, and splitting sites on the group I intron are located at positions 18-1020 of a nucleotide sequence as shown in SEQ ID NO: 2.

4. The construct for in vitro RNA synthesis and circularization according to claim 1, wherein a nucleotide sequence of the 3′-intron is as shown in SEQ ID NO: 29, a nucleotide sequence of the 5′-intron is as shown in SEQ ID NO: 6, and lengths of exon 1 and exon 2 are 0 nt.

5. The construct for in vitro RNA synthesis and circularization according to claim 1, wherein the construct comprises a T7 promoter, and the T7 promoter is located at the 5′ end of the 3′-intron.

6. The construct for in vitro RNA synthesis and circularization according to claim 5, wherein a nucleotide sequence of the T7 promoter is as shown in SEQ ID NO: 11.

7. The construct for in vitro RNA synthesis and circularization according to claim 1, wherein the construct comprises a CVB3 IRES sequence, the CVB3 IRES sequence is located between the gene of interest and the exon 1.

8. The construct for in vitro RNA synthesis and circularization according to claim 7, wherein a nucleotide sequence of the CVB3 IRES sequence is as shown in SEQ ID NO: 13.

9. A method for constructing a construct for in vitro RNA synthesis, wherein the method comprises: performing a structural analysis on a group I intron of a cob gene of Fusarium oxysporum to obtain splitting sites related to an enzyme activity of the group I intron, and constructing a construct framework; and introducing a gene of interest into the construct framework to complete the construct.

10. The method for constructing a construct for in vitro RNA synthesis according to claim 9, wherein a nucleotide sequence of the group I intron is as shown in SEQ ID NO: 2.

11. The method for constructing a construct for in vitro RNA circularization according to claim 9, wherein the splitting sites are at positions 18-1020 of the nucleotide sequence as shown in SEQ ID NO: 2.

12. A construct obtained by the method for constructing a construct for in vitro RNA synthesis according to claim 9.

13. The construct for in vitro RNA circularization according to claim 12, wherein the construct comprises 3′-intron, exon 2, a gene of interest, exon 1, and 5′-intron in sequence following the 5′-3′ direction.

14. The construct for in vitro RNA circularization according to claim 13, wherein a nucleotide sequence of the 3′-intron is as shown in SEQ ID NO: 29, a nucleotide sequence of the 5′-intron is as shown in SEQ ID NO: 6, and lengths of exon 1 and exon 2 are 0 nt.

15. A method for RNA circularization, comprising taking the construct according to claim 1 for transcription to obtain a linear RNA molecule; and inducing the linear RNA molecule for circularization to obtain the circular RNA.

16. A circular RNA, being prepared by the method for RNA circularization according to claim 15.

17. Applications of the circular RNA according to claim 16 in the preparation of medicines, vaccines and/or cells highly expressing target genes.


SEQUENCE LISTING (38 sequences)

SEQ ID NO: 1 - DNA, 2410 bp, Fusarium oxysporum
  source 1..2410: mol_type = genomic DNA
atgagaatattaaaaaatcaccctttattaaaattagctaatggttatttaatagacgcttcacaacctagtaatataagttacttatgaaatttcggatctttattattagtttgtttagttatacaaattgtaactggagttactttagctatgcattataacccttcagtattagaagcatttaattctgtagaacatattatgcgtgatgtaaacaatggatgattagtacgttacttacatagtaacacagcatcagctttcttctttttagtgtacttacacataggaagaggtatatattacgcatcttatagagcaccaagaacattgacatgagttataggtacaataatccttatcgttatgatagttacaggattcctgggttaccatacaatagcccaaaacgactataataataataaaataacaactacaacaacctttaacgataaaagatattattcaacatcgagaaataatgaagaggagacttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgttttaccttatggacaaatgtcattatgaggtgctacagttattactaatttaattagtgctgttccttgaattggacaagacatagttgagttcatttgaggaggttttagcgtaaataacgcaactttaaacagattctttgctttacattttgtattaccttttatattagcggcattagttttaatgcatatgatagctttacacgatacagctggatcaagtaatcctttaggagtaccagtatattatgatagaatgcctatggctccatatttcttatttaaagatttaataactatatttatctttatatttgttttaggtatctttgtattctttatgcctaatgttttaggtgatagtgataattatataatggctaaccctatgcaaacaccacctgcgatagtacctgaatgatatttattacctttctatgctattttaagatctatccctaataaattattaggagttatcgcaatgttagctgcaatactaattattttactattacctaaagctgatttagggttaactaaaggtttacaatttagacccctaagcaagatagctttctacctgtttttagtgaattttttaatgttaatgcaattaggggctaaacacgtagaaagcccgtttatagaaataggacaattaagtactgcgttatatttttctcatttcttatttatattaccggcagtaagtatattagaaaatactcttgtagatttagaattacaaaataatgccaaaaatatataa

SEQ ID NO: 2 - DNA, 1237 bp, Fusarium oxysporum
  source 1..1237: mol_type = genomic DNA
taccatacaatagcccaaaacgactataataataataaaataacaactacaacaacctttaacgataaaagatattattcaacatcgagaaataatgaagaggagacttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttg

SEQ ID NO: 3 - DNA, 1136 bp, synthetic construct
  source 1..1136: mol_type = other DNA
ttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgtt

SEQ ID NO: 4 - DNA, 112 bp, synthetic construct
  source 1..112: mol_type = other DNA
tgggttaccatacaatagcccaaaacgactataataataataaaataacaactacaacaacctttaacgataaaagatattattcaacatcgagaaataatgaagaggagac

SEQ ID NO: 5 - DNA, 1100 bp, synthetic construct
  source 1..1100: mol_type = other DNA
aaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgtt

SEQ ID NO: 6 - DNA, 148 bp, synthetic construct
  source 1..148: mol_type = other DNA
tgggttaccatacaatagcccaaaacgactataataataataaaataacaactacaacaacctttaacgataaaagatattattcaacatcgagaaataatgaagaggagacttcgttctcacgtataaataagtttttattagccaa

SEQ ID NO: 7 - DNA, 507 bp, synthetic construct
  source 1..507: mol_type = other DNA
taaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgtt

SEQ ID NO: 8 - DNA, 741 bp, synthetic construct
  source 1..741: mol_type = other DNA
tgggttaccatacaatagcccaaaacgactataataataataaaataacaactacaacaacctttaacgataaaagatattattcaacatcgagaaataatgaagaggagacttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaat

SEQ ID NO: 9 - DNA, 80 bp, synthetic construct
  source 1..80: mol_type = other DNA
cgcatcttatagagcaccaagaacattgacatgagttataggtacaataatccttatcgttatgatagttacaggattcc

SEQ ID NO: 10 - DNA, 80 bp, synthetic construct
  source 1..80: mol_type = other DNA
ttaccttatggacaaatgtcattatgaggtgctacagttattactaatttaattagtgctgttccttgaattggacaaga

SEQ ID NO: 11 - DNA, 20 bp, synthetic construct
  source 1..20: mol_type = other DNA
taatacgactcactataggg

SEQ ID NO: 12 - DNA, 687 bp, synthetic construct
  source 1..687: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgataa

SEQ ID NO: 13 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 14 - DNA, 2862 bp, synthetic construct
  source 1..2862: mol_type = other DNA
taatacgactcactatagggttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgttttaccttatggacaaatgtcattatgaggtgctacagttattactaatttaattagtgctgttccttgaattggacaagagccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgataattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaacgcatcttatagagcaccaagaacattgacatgagttataggtacaataatccttatcgttatgatagttacaggattcctgggttaccatacaatagcccaaaacgactataataataataaaataacaactacaacaacctttaacgataaaagatattattcaacatcgagaaataatgaagaggagac

SEQ ID NO: 15 - DNA, 2862 bp, synthetic construct
  source 1..2862: mol_type = other DNA
taatacgactcactatagggaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgttttaccttatggacaaatgtcattatgaggtgctacagttattactaatttaattagtgctgttccttgaattggacaagagccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgataattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaacgcatcttatagagcaccaagaacattgacatgagttataggtacaataatccttatcgttatgatagttacaggattcctgggttaccatacaatagcccaaaacgactataataataataaaataacaactacaacaacctttaacgataaaagatattattcaacatcgagaaataatgaagaggagacttcgttctcacgtataaataagtttttattagccaa

SEQ ID NO: 16 - DNA, 2882 bp, synthetic construct
  source 1..2882: mol_type = other DNA
taatacgactcactatagggtcatccttacatattcaacgtaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtatgttttaccttatggacaaatgtcattatgaggtgctacagttattactaatttaattagtgctgttccttgaattggacaagagccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgataattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaacgcatcttatagagcaccaagaacattgacatgagttataggtacaataatccttatcgttatgatagttacaggattcctgggttaccatacaatagcccaaaacgactataataataataaaataacaactacaacaacctttaacgataaaagatattattcaacatcgagaaataatgaagaggagacttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaat

SEQ ID NO: 17-18 - skipped (2 entries)

SEQ ID NO: 19 - DNA, 10 bp, synthetic construct
  source 1..10: mol_type = other DNA
acaggattcc

SEQ ID NO: 20 - DNA, 10 bp, synthetic construct
  source 1..10: mol_type = other DNA
ttaccttatg

SEQ ID NO: 21 - DNA, 20 bp, synthetic construct
  source 1..20: mol_type = other DNA
tatgatagttacaggattcc

SEQ ID NO: 22 - DNA, 20 bp, synthetic construct
  source 1..20: mol_type = other DNA
ttaccttatggacaaatgtc

SEQ ID NO: 23 - DNA, 30 bp, synthetic construct
  source 1..30: mol_type = other DNA
tccttatcgttatgatagttacaggattcc

SEQ ID NO: 24 - DNA, 30 bp, synthetic construct
  source 1..30: mol_type = other DNA
ttaccttatggacaaatgtcattatgaggt

SEQ ID NO: 25 - DNA, 40 bp, synthetic construct
  source 1..40: mol_type = other DNA
ggtacaataatccttatcgttatgatagttacaggattcc

SEQ ID NO: 26 - DNA, 40 bp, synthetic construct
  source 1..40: mol_type = other DNA
ttaccttatggacaaatgtcattatgaggtgctacagtta

SEQ ID NO: 27 - DNA, 50 bp, synthetic construct
  source 1..50: mol_type = other DNA
atgagttataggtacaataatccttatcgttatgatagttacaggattcc

SEQ ID NO: 28 - DNA, 50 bp, synthetic construct
  source 1..50: mol_type = other DNA
ttaccttatggacaaatgtcattatgaggtgctacagttattactaattt

SEQ ID NO: 29 - DNA, 1094 bp, synthetic construct
  source 1..1094: mol_type = other DNA
aaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttg

SEQ ID NO: 30 - DNA, 141 bp, synthetic construct
  source 1..141: mol_type = other DNA
ccatacaatagcccaaaacgactataataataataaaataacaactacaacaacctttaacgataaaagatattattcaacatcgagaaataatgaagaggagacttcgttctcacgtataaataagtttttattagccaa

SEQ ID NO: 31 - DNA, 1098 bp, synthetic construct
  source 1..1098: mol_type = other DNA
aaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttgtgtt

SEQ ID NO: 32 - DNA, 146 bp, synthetic construct
  source 1..146: mol_type = other DNA
tgggtccatacaatagcccaaaacgactataataataataaaataacaactacaacaacctttaacgataaaagatattattcaacatcgagaaataatgaagaggagacttcgttctcacgtataaataagtttttattagccaa

SEQ ID NO: 33 - DNA, 148 bp, synthetic construct
  source 1..148: mol_type = other DNA
tcccttaccatacaataggggaaaacgactataataataataaaataacaactacaacaacctttaacgataaaagatattattcaacatcgagaaataatgaagaggagacttcgttctcacgtataaataagtttttattagccaa

SEQ ID NO: 34 - DNA, 148 bp, synthetic construct
  source 1..148: mol_type = other DNA
taaattaccatacaatagtttaaaacgactataataataataaaataacaactacaacaacctttaacgataaaagatattattcaacatcgagaaataatgaagaggagacttcgttctcacgtataaataagtttttattagccaa

SEQ ID NO: 35 - DNA, 148 bp, synthetic construct
  source 1..148: mol_type = other DNA
ttttttaccatacaatagaaaaaaacgactataataataataaaataacaactacaacaacctttaacgataaaagatattattcaacatcgagaaataatgaagaggagacttcgttctcacgtataaataagtttttattagccaa

SEQ ID NO: 36 - DNA, 1130 bp, synthetic construct
  source 1..1130: mol_type = other DNA
ttcgttctcacgtataaataagtttttattagccaaaaatcttaatcctgtttttatctatcataatctaaatgaagattcagttcgtagaaatatagctaaagaaactaagggacttagtggtatttatatgatcttaaataaagaaactttaagttattatattggatcagcttctactgacagaattaactctagattttcaaaacatttaatatatttaaatggtagtaaaatagttaaaaattcagttaataaatatggtttacataattttgcctttattgtattagaattattccctgaaatagttaatcaagaaaataataaaaaattattagatttagaagatttttatctaaaatctcttttacctgactataatatattaaccgaagcaggatctagctttggatataaacatactgaagttaatagaataaagatgaaagctaattatagtgagaagcgtagagaagaaataggtagtttgaatagaggtaaaactttatcttctgaaactatagaaactatgagacaatcagctttaaatagaaaacctttagactatacagaacaaggtgttttaaatatgaaaaagaattctaagcctattatagtaaaagaattaaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttg

SEQ ID NO: 37 - DNA, 501 bp, synthetic construct
  source 1..501: mol_type = other DNA
taaataatactgtatatggcgaatttaatagtatagttgaagcagcagaagctttaaattgttcaactaaaactatacaaagaacattaaaaactcctagcaaaacattaaaaggacgttgaatagttgattattttaaataaggggcaagctctaaaataaattattattatagttgtagtcctgcgagtataaagttttatgcaatttatggaaagaaataaaacttaaagcagaataacctgacaaggttattgaagaaacctaatagtttctttggcaatattagtgaaaacgatcaaaatatattgtaactacaatataagagatcgtcggttatccatataatcgcgacagactgggtcactaatgggtggctgaaatgctgcttaatgcacagtcgaaactttatatcaaggcgatatatatcgctctaatataggtgataaacgaacaataaagttatcatcgcttttgtactttgctacaaaagtaagtttg

SEQ ID NO: 38 - DNA, 22 bp, synthetic construct
  source 1..22: mol_type = other DNA
tgggttaccatacaatagccca